(19) United States
(12) Patent Application Publication
(10) Pub. No.: US 2024/0141316 A1
(43) Pub. Date: May 2, 2024

(54) METHODS AND KITS FOR ISOLATING TARGET NUCLEIC ACIDS BELOW A TARGET SIZE FROM A SAMPLE

(71) Applicant: Phase Scientific International, Ltd., Hong Kong (CN)

(72) Inventors: Vasu SAINI, Hong Kong (CN); Cheuk Yiu Tenny CHUNG, Hong Kong (CN); Daniel William BRADBURY, Hong Kong (CN); Harsha Madan KITTUR, Singapore (SG); Cheuk Yin LAM, Hong Kong (CN); Kar Kee TSE, Hong Kong (CN); Kit CHEUNG, Hong Kong (CN); Yin To CHIU, Hong Kong (CN); Garrett Lee MOSLEY, Hong Kong (CN)

(21) Appl. No.: 18/463,312

(22) Filed: Sep. 8, 2023

Related U.S. Application Data
(60) Provisional application No. 63/381,933, filed on Nov. 2, 2022.

(51) Int. Cl. C12N 15/10; B03C 1/01; C07H 1/06

(52) U.S. Cl. CPC C12N 15/1013; B03C 1/01; C07H 1/06

(57) ABSTRACT

The present disclosure relates to methods, compositions, and kits for isolating target nucleic acids below a target size from a sample comprising nucleic acid components. In some embodiments, the methods involve one or more aqueous two-phase system (ATPS) compositions, at least one solid phase medium, and at least one buffer. Some embodiments provide a kit comprising one or more ATPS compositions, at least one solid phase medium, and at least one buffer. Other embodiments provide methods for diagnosing a disease or condition using the methods described herein.

### CROSS-REFERENCE TO RELATED APPLICATION

This application claims priority to, and the benefits of, U.S. Provisional Application having Ser. No. 63/381,933 filed on Nov. 2, 2022. The entire contents of the foregoing application are hereby incorporated by reference in its entirety for all purposes.

### FIELD OF INVENTION

This application relates to methods and kits for isolating target nucleic acids. More specifically, the present application relates to methods and kits for isolating target nucleic acids below a target size from a sample.

### BACKGROUND OF INVENTION

It is a challenge to efficiently concentrate and isolate target nucleic acids below certain size from samples, especially to isolate, purify, and concentrate the very rare and sparse nucleic acid fragments against a complex background of biological matrix. Oftentimes the yield of relevant fragments is so low that subsequent analysis may not have sufficient diagnostic sensitivity and specificity. For certain applications such as non-invasive prenatal testing (NIPT) and circulating tumor DNA enrichment, difference in the size is an important criterion to distinguish target nucleic acids from non-target nucleic acids. Accordingly, there is a need for improved methods that are simple, stable, robust, and efficient in isolating target nucleic acids below a certain size from samples.

### SUMMARY OF INVENTION

Disclosed herein are novel methods and kits that are useful for isolation, concentration and/or purification of target analytes, such as nucleic acids, that are below a target size, employing solid phase media such as beads or columns.

In some embodiments, provided is a method for isolating target nucleic acids below a target size from a sample including nucleic acid components; including the steps of: (a) preparing a sample solution from the sample; (b) contacting a plurality of beads with the sample solution, wherein the nucleic acid components bind to the plurality of beads to form a beads-analyte complex; (c) mixing the beads-analyte complex with a fractionation buffer including at least one chaotropic agent to form a bulk fractionation solution, wherein the target nucleic acids below the target size are released from the beads-analyte complex into the bulk fractionation solution; (d) immobilizing the beads-analyte complex; and (e) separating the bulk fractionation solution including the isolated target nucleic acids below the target size from the immobilized beads-analyte complex.

In some embodiments, provided is a kit for isolating target nucleic acids below a target size from a sample including nucleic acid components, including: (a) at least one ATPS components selected from the group consisting of a polymer, salt, surfactant, and combinations thereof; (b) a plurality of beads; (c) a fractionation buffer including at least one chaotropic agent selected from the group consisting of thiocyanate, isothiocyanate, perchlorate, acetate, trichloroacetate, trifluoroacetate, chloride, and iodide; and (d) a binding buffer includes at least one chaotropic agent selected from the group consisting of thiocyanate, isothiocyanate, perchlorate, acetate, trichloroacetate, trifluoroacetate, chloride, and iodide.

In some embodiments, provided is a method for concentrating and purifying one or more target analytes from a sample solution, comprising the steps of:
(a) adding a sample solution containing the target analyte(s) to a first aqueous two-phase system (ATPS) to form a mixture that partitions into a first phase and a second phase, wherein the target analyte(s) are concentrated in the first phase;
(b) isolating the first phase containing the concentrated target analyte(s), thereby resulting in a concentrated solution;
(c) applying magnetic beads to the concentrated solution, such that the magnetic beads bind the target analyte(s) to form a beads-analyte complex; and
(d) recovering the target analyte(s) from the beads-analyte complex, resulting in a final solution containing the target analyte(s) that is concentrated and purified.

In some embodiments, a respective kit can be advantageously used in conjunction with and for performing the methods according to the various aspects of the invention. In some embodiments, the kit may include the components described in the various embodiments, but may additionally include syringe or pipette accessible containers for storage, packing, and/or reactions and optionally equipment for manipulating the aqueous solutions. Such containers and equipment may include columns, test tubes, capillary tubes, plastic test tubes, falcon tubes, culture tubes, well plates, pipettes, cuvettes or the like.

Other example embodiments are discussed herein.

### Advantages

There are many advantages to the various embodiments of the present disclosure. For instance, the methods and kits of the present disclosure surprisingly and effectively concentrate and purify target analytes below a target size from samples such as clinical/biological samples. The methods and kits are particularly effective at purifying target analytes that exist at very small concentrations in the biological sample, such as cell-free DNA. As is shown by the examples, the methods of the present disclosure allow for a precise size selection of the recovered DNA molecules.

In some embodiments, the methods of the present disclosure (termed as “reverse fractionation” in some embodiments) utilize total binding of nucleic acids components in the sample followed by selective unbinding of target nucleic acids below a target size from solid phase medium (such as a magnetic beads-analyte complex, or solid phase extraction column). The disclosed methods achieve a surprisingly improved nucleic acid size fractionation compared to methods that utilize a first binding step to selectively bind and remove unwanted larger nucleic acids, leaving the target nucleic acids below a target size in the supernatant to be further purified by a solid phase medium.

In certain embodiments, the disclosed methods can accommodate variations in the volume of samples and can surprisingly achieve a stable and efficient DNA size fractionation across different sample volumes, with a stable DNA cutoff value and recovery of DNA, especially small DNA fragments. In certain embodiments, the disclosed methods are compatible with different sample types (such as plasma and urine), and demonstrate stable and efficient DNA size fractionation across different sample types. This allows for broad applications and analysis, such as diagnosis of diseases or conditions that require different types of clinical/biological samples.

Purified nucleic acids obtained by the disclosed methods can be subject to a wide range of downstream applications such as detection or analysis of the nucleic acids in forensic, diagnostic or therapeutic applications, and laboratory procedures such as sequencing, amplification, reverse transcription, labeling, digestion, blotting procedures and the like. The disclosed methods can improve the performance of downstream characterization or processing of the nucleic acids.

In certain embodiments, the disclosed methods and kits can be used in a wide variety of applications. For example, the methods and kits of the present disclosure can be used for size selective fractionation of DNA during the preparation of a sequencing library, i.e. to isolate DNA molecules of a desired size or size range to be used for the subsequent sequencing applications, such as next generation sequencing (NGS). In certain embodiments, the disclosed methods and kits can be used for enriching fetal fraction in a maternal sample for non-invasive prenatal testing (NIPT) by effectively isolating fetal nucleic acids from maternal nucleic acids. In certain embodiments, the disclosed methods and kits can be used to increase the ratio of circulating tumor DNA:cell-free DNA, and/or variant allele frequency (VAF) in the clinical sample for further analysis, such as cancer diagnostic assay.

These and other features and characteristics, as well as the methods of operation and functions of the related components, will become more apparent upon consideration of the following detailed description and the appended claims with reference to the accompanying figures, all of which form a part of this specification, wherein like reference numerals designate corresponding parts in the various figures. It is to be expressly understood, however, that the drawings are for the purpose of illustration and description only and are not intended as a definition of the limits of the claims.

### BRIEF DESCRIPTION OF FIGURES

FIG. 1A shows an example workflow of direct fractionation according to an example embodiment.

FIG. 1B shows an example workflow of reverse fractionation according to an example embodiment.

FIG. 2A shows an electropherogram of DNA oligos recovery of plasma extracted from different volumes of top phase in the 2^{nd} ATPS using direct fractionation with around 150 bp expected DNA cutoff value according to an example embodiment.

FIG. 2B shows an electropherogram of DNA oligos recovery of plasma extracted from different volumes of top phase in the 2^{nd} ATPS using reverse fractionation with around 150 bp expected DNA cutoff value according to an example embodiment.

FIG. 3A shows an electropherogram of DNA oligos recovery of plasma extracted from different volumes of top phase in the 2^{nd} ATPS using direct fractionation with around 300 bp expected DNA cutoff value according to an example embodiment.

FIG. 3B shows an electropherogram of DNA oligos recovery of plasma extracted from different volumes of top phase in the 2^{nd} ATPS using reverse fractionation with around 300 bp expected DNA cutoff value according to an example embodiment.

FIG. 4A shows an electropherogram of DNA oligos recovery of different sample types using direct fractionation with around 150 bp expected DNA cutoff value according to an example embodiment.

FIG. 4B shows an electropherogram of DNA oligos recovery of different sample types using reverse fractionation with around 150 bp expected DNA cutoff value according to an example embodiment.

FIG. 5A shows an electropherogram of DNA oligos recovery of different sample types using direct fractionation with around 300 bp expected DNA cutoff value according to an example embodiment.

FIG. 5B shows an electropherogram of DNA oligos recovery of different sample types using reverse fractionation with around 300 bp expected DNA cutoff value according to an example embodiment.

FIGS. 6A-6H show electropherograms of DNA oligos recovery of plasma sample using reverse fractionation with different reverse fractionation buffer formulas (Buffer R-015 to Buffer R-021 respectively) according to example embodiments.

### DETAILED DESCRIPTION

Unless indicated otherwise, the terms used herein, including technical and scientific terms, have the same meaning as usually understood by those skilled in the art to which the present invention pertains.

As used herein and in the claims, the terms “comprising” (or any related form such as “comprise” and “comprises”), “including” (or any related forms such as “include” or “includes”), “containing” (or any related forms such as “contain” or “contains”), or “having” (or any related forms such as “have” or “has”) means including the following elements but not excluding others. It shall be understood that for every embodiment in which the term “comprising” (or any related form such as “comprise” and “comprises”), “including” (or any related forms such as “include” or “includes”), or “containing” (or any related forms such as “contain” or “contains”) is used, this disclosure/application also includes alternate embodiments where the term “comprising”, “including,” “containing,” or “having” is replaced with “consisting essentially of” or “consisting of”. These alternate embodiments that use “consisting of” or “consisting essentially of” are understood to be narrower embodiments of the “comprising”, “including,” or “containing,” embodiments.

For example, alternate embodiments of “a solution comprising A, B, and C” would be “a solution consisting of A, B, and C” and “a solution consisting essentially of A, B, and C.” Even if the latter two embodiments are not explicitly written out, this disclosure/application includes those embodiments. Furthermore, it shall be understood that the scopes of the three embodiments listed above are different.

For the sake of clarity, “comprising”, including, and “containing”, and any related forms are open-ended terms which allows for additional elements or features beyond the named essential elements, whereas “consisting of” is a closed end term that is limited to the elements recited in the claim and excludes any element, step, or ingredient not specified in the claim.

“Essentially consisting of” limits the scope of a claim to the specified materials, components, or steps (“essential elements”) that do not materially affect the essential characteristic(s) of the claimed invention. In some embodiments, the essential characteristics are the basic and novel characteristic(s) of the claimed invention.

As used herein, the singular forms “a”, “an” and “the” are intended to include the plural forms as well, unless the context clearly indicates otherwise. In some embodiments, the terms “a” and “an” are interchangeable with terms such as “at least one” and “one or more”.

Where a range is referred in the specification, the range is understood to include at least each discrete point within the range. For example, 1-7 in some embodiments means 1, 2, 3, 4, 5, 6, and 7. Unless otherwise indicated, a range is meant to include all values that fall within the range, including whole numbers, fractions, portions, and the like. For example, a range of 1-7 when described in a claim refers to a scope that includes values and sub-ranges such as 1, 1.5, 2-3, 6, and 7, by way of example.

As used herein, the term “about” is understood as within a range of normal tolerance in the art and not more than ±10% of a stated value. By way of example only, about 50 means from 45 to 55 including all values in between. As used herein, the phrase “about” a specific value also includes the specific value, for example, about 50 includes 50.

“Aqueous”, as used herein, refers to the characteristic properties of a solvent/solute system wherein the solvating substance has a predominantly hydrophilic character. Examples of aqueous solvent/solute systems include those where water, or compositions containing water, are the predominant solvent. The polymer and/or surfactant components whose use is described in the embodiments are “aqueous” in the sense that they form aqueous phases when combined with a solvent such as water. Further, as understood by the skilled person, in the present context the term liquid “mixture” refers merely to a combination of the herein-defined components.

As used herein, an aqueous two-phase system (ATPS) means a liquid-liquid separation system that can accomplish isolation or concentration of an analyte by partitioning, where two phases, sections, areas, components, or the like, interact differently with at least one analyte to which they are exposed and optionally dissolved. An ATPS is formed when two immiscible phase forming components, such as a salt and polymer, or two incompatible polymers (e.g., PEG and dextran) with certain concentration are mixed in an aqueous solution. ATPS methods are relatively inexpensive and scalable because they employ two-phase partitioning to separate analytes (e.g., nucleic acids) from contaminants.

The term “isolated” as used herein refers to an analyte being removed from its original environment and thus is altered from its original environment. For example, an isolated nucleic acid generally is provided with fewer non-nucleic acid components (e.g., protein, lipid) than the amount of components present in a source sample. A composition comprising an isolated analyte, (e.g., sample nucleic acid) can be substantially isolated (e.g., about 90%, 91%, 92%, 93%, 94%, 95%, 96%, 97%, 98%, 99% or greater than 99% free of non-analyte components (such as non-nucleic acid components)).

As used herein, “concentrated” means that the mass ratio of analyte in question to the solution in which the analyte is suspended is higher than the mass ratio of said analyte in its pre-concentration solution. It can, for example, be slightly higher, or more preferably at least twice, ten times or one hundred times as high.

As used herein, “biological sample” refers to any tangible material obtained directly or indirectly from an organism, such as a virus, bacterium, plant, animal, or human Examples of biological samples include but are not limited to nucleic acids, proteins, cells, cellular organelles, tissue extracts, tissues, organs, biofluids such as blood, plasma, urine, saliva, stool, cerebrospinal fluid (CSF), lymph, serum, sputum, peritoneal fluid, sweat, tears, nasal swab, vaginal swab, endocervical swab, semen, breast milk, and other bodily fluids.

As used herein, “clinical sample” refers to any sample obtained directly or indirectly from a subject (e.g., a human). In some embodiments the subject is a human patient. Examples of clinical samples include but are not limited to blood, plasma, urine, saliva, stool, cerebrospinal fluid (CSF), lymph, serum, sputum, peritoneal fluid, sweat, tears, nasal swab, vaginal swab, endocervical swab, semen, breast milk, and other bodily fluids.

As used herein, “nucleic acid components” refers generally to the nucleic acids extracted from a given sample regardless of their size. In some embodiments, nucleic acid components include DNA, RNA or combinations thereof. Examples of nucleic acid components include, but not limited to, gDNA, cDNA, plasmid DNA, mitochondrial DNA, cell-free DNA (cfDNA), circulating tumor DNA (ctDNA), circulating fetal DNA, cell-free microbial DNA, micro RNA (miRNA), messenger RNA (mRNA), transfer RNA (tRNA), ribosomal RNA (rRNA), circular RNA, long non-coding RNA (lncRNA) or combinations thereof.

In some embodiments, the “target nucleic acid”, “target analyte”, or “small DNA fragment” refers to a nucleic acid fragment below a selected size, e.g., a nucleic acid comprising fewer than 1000 basepairs (e.g., fewer than 1000 bp, 900 bp, 800 bp, 700 bp, 600 bp, 500 bp, 450 bp, 400 bp, 350 bp, 300 bp, 250 bp, 200 bp, 150 bp, 100 bp, or 50 bp.) In some embodiments, the target nucleic acids/analyte is single-stranded nucleic acid, while in other embodiments the target nucleic acids/analyte is a double-stranded nucleic acid. In some embodiments, the target nucleic acids/analyte is DNA or RNA. Examples of target nucleic acids/analyte include, but not limited to, gDNA, cDNA, plasmid DNA, mitochondrial DNA, cell-free DNA (cfDNA), circulating tumor DNA (ctDNA), circulating fetal DNA, cell-free microbial DNA, micro RNA (miRNA), messenger RNA (mRNA), transfer RNA (tRNA), ribosomal RNA (rRNA), circular RNA, long non-coding RNA (lncRNA) or combinations thereof.

As used herein, “cell-free DNA” (cfDNA) is DNA that is present outside a cell, e.g., DNA present in the sample (e.g. blood, plasma, serum, or urine) obtained from a subject.

As used herein, the term “polymer” refers to any polymer including at least one substituted or non-substituted monomer. Examples of “polymer” include, but are not limited to, homopolymer, copolymer, terpolymer, random copolymer, and block copolymer. Block copolymers include, but are not limited to, block, graft, dendrimer, and star polymers.

As used herein, “copolymer” refers to a polymer derived from two monomeric species; similarly, a terpolymer refers to a polymer derived from three monomeric species. The polymer also includes various morphologies, including, but not limited to, linear polymer, branched polymer, random polymer, crosslinked polymer, and dendrimer systems. In some embodiments, a polymer also includes its chemically modified equivalent, such as hydrophobically-modified, or silicone-modified. As an example, polyacrylamide polymer refers to any polymer including at least one substituted or non-substituted acrylamide unit, e.g., a homopolymer, copolymer, terpolymer, random copolymer, block copolymer or terpolymer of polyacrylamide; polyacrylamide can be a linear polymer, branched polymer, random polymer, crosslinked polymer, or a dendrimer of polyacrylamide; polyacrylamide can be hydrophobically-modified polyacrylamide, or silicone-modified polyacrylamide.

In some embodiments, examples of polymers include, but are not limited to, polyethers, polyimines, polyalkylene glycols, alkoxylated surfactants, polysaccharides, polyether-modified silicones, polyacrylamides, polyacrylic acids and copolymers thereof. In some embodiments, the polymer is hydrophobically-modified, or silicone-modified.

Examples of polyalkylene glycols (also referred as ‘PAG’ or ‘poly(oxyalkylene)’ or ‘poly(alkylene oxide)’) include, but are not limited to, hydrophobically modified polyalkylene glycols, poly(oxyalkylene)polymer, poly(oxyalkylene)copolymer, hydrophobically modified poly(oxyalkylene)copolymers, dipropylene glycol, tripropylene glycol, polyethylene glycol (also referred as ‘PEG’), polypropylene glycol (also referred as ‘PPG’). In some embodiments, examples of copolymers of PAGs include, but are not limited to, poly(ethylene glycol-propylene glycol) (also referred as ‘PEG-PPG’ or ‘UCON’), and poly(ethylene glycol-ran-propylene glycol) (also referred as TEG-ran-PPG′). In some embodiments, PEG-PPG comprises random copolymers, block copolymers, or combination thereof. In some embodiments, PEG-PPG comprise both random copolymers and block copolymers. In some embodiments, PEG-PPG is PEG-ran-PPG.

As used herein, “vinyl polymer” refers to a group of polymers derived from substituted vinyl (H₂C═CHR) monomers. Examples of vinyl polymer include, but are not limited to, polyvinyl pyrrolidone, polyvinyl alcohol, polyvinyl caprolactam, and polyvinyl methylether.

Examples of polysaccharides include, but are not limited to, dextran, carboxymethyl dextran, dextran sulfate, hydroxypropyl dextran, starch, carboxymethyl cellulose, hydroxypropyl cellulose, methyl cellulose, ethylhydroxyethylcellulose, and maltodextrin. In some embodiments, polysaccharides are alkoxylated starches, alkoxylated cellulose, or alkyl hydroxyalkyl cellulose.

Examples of polyacrylamides include, but are not limited to, poly N-isopropylacrylamide.

Examples of polyimines include, but are not limited to, polyethyleneimine.

Examples of alkoxylated surfactants include, but are not limited to, carboxylates, sulphonates, petroleum sulphonates, alkylbenzenesulphonates, naphthalenesulphonates, olefin sulphonates, alkyl sulphates, sulphates, sulphated natural oils, sulphated natural fats, sulphated esters, sulphated alkanolamides, sulphated alkylphenols, ethoxylated alkylphenols, sodium N-lauroyl sarcosinate (NLS), ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, and polyoxyethylene fatty acid amides.

In some embodiments, the polymer has an average molecular weight of about 200-1,000 Da, 200-35,000 Da, 300-35,000 Da, 400-2,000 Da, or 400-35,000 Da. Examples thereof include, but are not limited to, polyalkylene glycols (PAGs) with average molecular weight of about 400 Da, 500 Da, 600 Da, 700 Da, 800 Da, 900 Da, 1,000 Da, 2,000 Da, 3,000 Da, 4,000 Da, 5,000 Da, 6,000 Da, 7,000 Da, 8,000 Da, 9,000 Da, 10,000 Da, 15,000 Da, 20,000 Da, 25,000 Da, 30000 Da, and 35000 Da. In some embodiments, the PAG has an average molecular weight at a range of between any of the two molecular weights listed above.

Examples of PAG include, but are not limited to PEG 200, PEG 300, PEG 400, PEG 500, PEG 600, PEG 700, PEG 800, PEG 900, PEG 1000, PEG 2000, PEG 3000, PEG 4000, PEG 5000, PEG 6000, PEG 7000, PEG 8000, PEG 9000, PEG 10000, PEG 15000, PEG 20000, PEG 25000, PEG 30000, PEG 35000, PPG 425, PPG 725, PPG 900, PPG 1000, and PPG 2000. In some embodiments, the PEG has an average molecular weight at a range of between any of the two PEG molecular weights listed above. In some embodiments, the PPG has an average molecular weight at a range of between any of the two PPG molecular weights listed above.

In some embodiments, the polymer comprises ethylene oxide (EO) and propylene oxide (PO) units, and has an ethylene oxide:propylene oxide (EO:PO) ratio of 90:10 to 10:90. In some embodiments, the polymer has an EO:PO ratio of 10:90, 15:85, 20:80, 25:75, 30:70, 35:65, 40:60, 45:55, 50:50, 55:45, 60:40, 65:35, 70:30, 75:25, 80:20, 85:15, or 90:10. In some embodiments, the polymer has an EO:PO ratio at a range between any of the two ratios listed above.

In some embodiments, the polymer is a PAG having an average molecular weight of about 980-12,000 Da and an EO:PO ratio of 50:50 to 75:25. Examples thereof include, but are not limited to, PEG-PPGs with average molecular weight of about 980 Da, 1,230 Da, 1,590 Da, 2,470 Da, 2,660 Da, 3,380 Da, 3,930 Da, 6,950 Da, and 12,000 Da. In some embodiments, the PEG-PPGs has an average molecular weight at a range of between any of the two PEG-PPGs molecular weights listed above. In some embodiments, PEG-PPG comprises an EO:PO ratio of 50:50, or 75:25. In some embodiments, the polymer is PEG-ran-PPG with an average molecular weight of about 2,500 or 12,000 Da and having an EO:PO ratio of about 75:25.

In some embodiments, the polymer is a vinyl polymer having an average molecular weight of about 2,500-2,500,000 Da. Examples thereof include, but are not limited to polyvinyl pyrrolidone with an average molecular weight of about 2,500 Da, 10,000 Da, 40,000 Da, 100,000 Da, and 2,500,000 Da. In some embodiments, the vinyl polymer has an average molecular weight at a range of between any of the two molecular weights listed above.

In some embodiments, the polymer is a polysaccharide and has an average molecular weight from about 6,000-5,000,000 Da. Examples thereof include, but are not limited to dextrans with average molecular weight of about 6,000 Da, 12,000 Da, 25,000 Da, 60,000 Da, 70,000 Da, 80,000 Da, 150,000 Da, 270,000 Da, 410,000 Da, 450,000 Da, 550,000 Da, 650,000 Da, 670,000 Da, 1,500,000 Da, 2,000,000 Da, 2,800,000 Da, 4,000,000 Da and 5,000,000 Da. In some embodiments, the dextran has an average molecular weight at a range of between any of the two molecular weights listed above.

In some embodiments, the polymer is a polyether and has an average molecular weight of about 200-35,000 Da. Examples thereof include, but are not limited to silicon modified polyether (or ‘polyether-modified silicones’) with average molecular weight of about 200 Da-35,000 Da.

In some embodiments, the polymer is a polyacrylamide and has an average molecular weight of 1,000-5,000,000 Da. Examples thereof include, but are not limited to polyacrylamide or poly(N-isopropylacrylamide) with average molecular weight of 1,000 Da, 2,000 Da, 5,000 Da, 10,000 Da, 40,000 Da, 85,000 Da, 5,000,000 Da. In some embodiments, the polyolefin has an average molecular weight at a range of between any of the two molecular weights listed above.

In some embodiments, the polymer is a polyacrylic acid and has an average molecular weight of about 1,250-4,000,000 Da. Examples thereof include, but are not limited to, polyacrylic acids with average molecular weight of 1,200 Da, 2,100 Da, 5,100 Da, 8,000 Da, 8,600 Da, 8,700 Da, 16,000 Da, and 83,000 Da. In some embodiments, the polyolefin has an average molecular weight at a range of between any of the two molecular weights listed above.

As used herein, the term “salt” refers to a substance containing a cation and an anion. Examples of salts include, but are not limited to, salts wherein the cation is sodium, potassium, calcium, ammonium, lithium, magnesium, aluminium, cesium, barium, straight or branched trimethyl ammonium, triethyl ammonium, tripropyl ammonium, tributyl ammonium, tetramethyl ammonium, tetraethyl ammonium, tetrapropyl ammonium or tetrabutyl ammonium, and/or wherein the anion is phosphate, hydrogen phosphate, dihydrogen phosphate, sulfate, sulfide, sulfite, hydrogen sulfate, carbonate, hydrogen carbonate, acetate, nitrate, nitrite, sulfite, chloride, fluoride, chlorate, perchlorate, chlorite, hypochlorite, bromide, bromate, hypobromite, iodide, iodate, cyanate, thiocyanate, isothiocyanate, oxalate, formate, chromate, dichromate, permanganate, hydroxide, hydride, citrate, borate, or tris. In some embodiments, the salts are kosmotropic salts, chaotropic salts, or inorganic salts.

In some embodiments, examples of “surfactant” include, but are not limited to, anionic surfactant, nonionic surfactant, cationic surfactant, zwitterion surfactant and amphoteric surfactant.

Examples of anionic surfactants include, but are not limited to, carboxylates, sulphonates, petroleum sulphonates, alkylbenzenesulphonates, naphthalenesulphonates, olefin sulphonates, alkyl sulphates, sulphates, sulphated natural oils & fats, sulphated esters, sulphated alkanolamides, ethoxylated alkylphenols, and sulphated alkylphenols.

Examples of nonionic surfactants include, but are not limited to, ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, and polyoxyethylene fatty acid amides.

Examples of cationic surfactants include, but are not limited to, quaternary ammonium salts, amines with amide linkages, polyoxyethylene alkyl & alicyclic amines, n,n,n′,n′ tetrakis substituted ethylenediamines, and 2-alkyl 1-hydroxethyl 2-imidazolines,

Examples of amphoteric surfactants include, but are not limited to, n-coco 3-aminopropionic acid/sodium salt, n-tallow 3-iminodipropionate, disodium salt, n-carboxymethyl n dimethyl n-9 octadecenyl ammonium hydroxide, n-cocoamidethyl n hydroxyethylglycine, and sodium salt, and sodium N-lauroyl sarcosinate (NLS).

In some embodiments, the surfactant comprises a polymer such as PAG. In some embodiments, the surfactant has a structure of EOₓ-PO_{y}-EOₓ, wherein EO refers to an ethylene oxide unit and PO refers to a propylene oxide unit, and x and y are the respective number of monomers. In some embodiments, x=2-136. In some embodiments, y=16-62. In some embodiments, examples of surfactants include, but are not limited to, (C₂H₄O)ₙC₁₄H₂₂O wherein n=4-10 (such as Triton X-100, Triton X-114, Triton X-45, Tween 20, Igepal CA630), Brij 58, Brij O10, Brij L23, EOx-POy-EOx wherein x=2-136 and y=16-62 (such as Pluronic L-61, Pluronic F-127), sodium dodecyl sulfate, sodium cholate, sodium cholate, sodium deoxycholate, N-lauroyl sarcosine sodium salt (NLS), hexadecyltrimethlammonium bromide, or span 80.

As used herein, the term “chaotropic agent” refers to a substance that disrupts the hydrogen bonding network between water molecules in a solution. For example, the chaotropic agent can include an anion selected from thiocyanate, isothiocyanate, perchlorate, acetate, trichloroacetate, trifluoroacetate, chloride, or iodide. The chaotropic agent can include a cation selected from sodium, guanidinium, lithium, or magnesium. Unless if otherwise indicated, a chaotropic agent defined by its cation or anion includes all compounds having an appropriate conjugate anion or cation, respectively. For example, 5M guanidinium would include guanidinium hydrochloride (GHCl), guanidinium thiocyanate, guanidinium isothiocyanate (GITC)) and the like. Examples of chaotropic agents include, but are not limited to, guanidinium hydrochloride (GHCl), guanidinium thiocyanate, guanidinium isothiocyanate (GITC)), sodium thiocyanate, sodium iodide, sodium perchlorate, sodium trichloroacetate, sodium trifluoroacetate, lithium perchlorate, lithium acetate, magnesium chloride, phenol, 2-propanol, thiourea, urea and the like.

The DNA sizes and cutoff values indicated herein with reference to basepairs “bp”, refer to the chain length of the DNA molecules and thus are used in order to describe the length of single-stranded as well as double-stranded DNA molecules. Thus, if the DNA is a single-stranded DNA molecule, it is understood that the above indications with respect to the size or length in “bp” refers to the size or length of nucleotides in said single-stranded DNA molecule.

### EMBODIMENTS OF THE PRESENT INVENTION

### Embodiment 1

One aspect provides a method for concentrating and purifying one or more target analytes from a sample solution, comprising the steps of:
(a) adding a sample solution containing the target analyte(s) to a first aqueous two-phase system (ATPS) to form a mixture that partitions into a first phase and a second phase, wherein the target analyte(s) are concentrated in the first phase;
(b) isolating the first phase containing the concentrated target analyte(s), thereby resulting in a concentrated solution;
(c) applying magnetic beads to the concentrated solution, such that the magnetic beads bind the target analyte(s) to form a beads-analyte complex; and
(d) recovering the target analyte(s) from the beads-analyte complex, resulting in a final solution containing the target analyte(s) that is concentrated and purified.

In some embodiments, the step (b) further comprises the following steps:
(i) adding the isolated first phase that is concentrated with the target analyte(s) to a second ATPS to form a second mixture that partitions into a third phase and a fourth phase, wherein the target analyte(s) are concentrated in the third phase; and
(ii) isolating the third phase containing the concentrated target analyte(s) to form the concentrated solution in step (b) for step (c).

In some embodiments, the concentrated solution of step (b) is mixed with a binding buffer, wherein the binding buffer comprises at least one chaotropic agent selected from n-butanol, ethanol, guanidinium chloride, guanidinium thiocyanate, lithium perchlorate, lithium acetate, magnesium chloride, phenol, 2-propanol, sodium dodecyl sulfate, thiourea, and urea, thereby resulting in the concentration solution for step (c).

In some embodiments, the step (d) further comprises the steps of:
(i) mixing the beads-analyte complex with a fractionation buffer comprising a polymer, a salt, a surfactant, a chaotropic agent or combinations thereof to form a fractionation solution, such that the target analyte(s) below a target size are released from the beads-analyte complex into the fractionation solution;
(ii) immobilizing the beads-analyte complex using a magnetic stand; and
(iii)isolating the target analyte(s) below the target size in the fractionation solution from the immobilized beads-analyte complex.

In some embodiments, the step (d) further comprises the steps of:
(iv) adding the isolated target analyte(s) below the target size to a second binding buffer, wherein the second binding buffer comprises at least one chaotropic agent selected from n-butanol, ethanol, guanidinium chloride, guanidinium thiocyanate, lithium perchlorate, lithium acetate, magnesium chloride, phenol, 2-propanol, sodium dodecyl sulfate, thiourea, and urea;
(v) applying magnetic beads to a mixture of the isolated target analyte(s) below the target size and the second binding buffer, wherein the magnetic beads bind the target analyte(s) below the target size to form a second beads-analyte complex; and
(vi) recovering the target analyte(s) from the second beads-analyte complex.

In some embodiments, the method further comprises the step of:
(e) subjecting said final solution to a diagnostic assay for detection and quantification of said target analyte(s).

In some embodiments, the target analyte(s) is selected from the group consisting of nucleic acids, a protein, an antigen, a biomolecule, a sugar moiety, a lipid, a sterol, and combinations thereof.

In some embodiments, the target analyte(s) is DNA.

In some embodiments, the target analyte(s) is cell-free DNA or circulating tumor DNA.

In some embodiments, the first ATPS comprises first ATPS components capable of forming the first phase and the second phase when the first ATPS components are dissolved in an aqueous solution, wherein the first ATPS components are selected from the group consisting of polymers, salts, surfactants, and combinations thereof.

In some embodiments, the second ATPS comprises second ATPS components capable of forming the third phase and the fourth phase when the second ATPS components are dissolved in an aqueous solution, wherein the second ATPS components are selected from the group consisting of polymers, salts, surfactants, and combinations thereof.

In some embodiments, the polymers dissolve in the aqueous solution at a concentration of 4%-84% (w/w).

In some embodiments, the salts dissolve in the aqueous solution at a concentration of 1%-80% (w/w). In some embodiments, the salts dissolve in the aqueous solution at a concentration of 8%-80% (w/w).

In some embodiments, the surfactants dissolve in the aqueous solution at a concentration of 0.05%-10% (w/w). In some embodiments, the surfactants dissolve in the aqueous solution at a concentration of 0.05%-9.8% (w/w).

In some embodiments, the step (a) further comprises the steps of:
(i) embedding a porous material with components capable of forming the first ATPS; and
(ii) contacting the sample solution with the porous material embedded with the components, wherein said components form the first phase and the second phase when the sample solution travels through said porous material.

In one aspect, provided is a method for concentrating and purifying one or more target analytes from a sample solution, comprising the steps of:
(a) adding a sample solution containing the target analyte(s) to a first aqueous two-phase system (ATPS) to form a mixture that partitions into a first phase and a second phase, wherein the target analyte(s) are concentrated in the first phase;
(b) isolating the first phase containing the concentrated target analyte(s);
(c) adding the isolated first phase that is concentrated with the target analyte(s) to a second ATPS to form a second mixture that partitions into a third phase and a fourth phase, wherein the target analyte(s) are concentrated in the third phase;
(d) isolating the third phase containing the concentrated target analyte(s), thereby resulting in a concentrated solution;
(e) mixing the concentration solution with a binding buffer, wherein the binding buffer comprises at least one chaotropic agent selected from n-butanol, ethanol, guanidinium chloride, guanidinium thiocyanate, lithium perchlorate, lithium acetate, magnesium chloride, phenol, 2-propanol, sodium dodecyl sulfate, thiourea, and urea;
(f) applying magnetic beads to the mixture of the concentrated solution and the binding buffer, such that the magnetic beads bind the target analyte(s) to form a beads-analyte complex; and
(g) mixing the beads-analyte complex with a fractionation buffer comprising a polymer, a salt, a surfactant, a chaotropic agent or combinations thereof to form a fractionation solution, such that the target analyte(s) below a target size are released from the beads-analyte complex into the fractionation solution;
(h) immobilizing the beads-analyte complex using a magnetic stand;
(i) isolating the target analyte(s) below the target size in the fractionation solution from the immobilized beads-analyte complex;
(j) adding the isolated target analyte(s) below the target size to a second binding buffer, wherein the second binding buffer comprises at least one chaotropic agent selected from n-butanol, ethanol, guanidinium chloride, guanidinium thiocyanate, lithium perchlorate, lithium acetate, magnesium chloride, phenol, 2-propanol, sodium dodecyl sulfate, thiourea, and urea;
(k) applying magnetic beads to a mixture of the isolated target analyte(s) below the target size and the second binding buffer, wherein the magnetic beads bind the target analyte(s) below the target size to form a second beads-analyte complex;
(l) recovering the target analyte(s) below the target size from the second beads-analyte complex, resulting in a final solution containing the target analyte(s) below the target size that is concentrated and purified; and
(m) subjecting said final solution to a diagnostic assay for detection and quantification of said target analyte(s) below the target size.

Various ATPS systems that can be used in various embodiments of the present invention include, but are not limited to, polymer-polymer, polymer-salt, polymer-surfactant, salt-surfactant, surfactant, surfactant-surfactant, or polymer-salt-surfactant.

In one embodiment, the first and/or second ATPS comprises a polymer. In some embodiments, possible polymers that may be employed include, but are not limited to, polyalkylene glycols (PAGs), such as hydrophobically modified polyalkylene glycols, poly(oxyalkylene)polymers, poly(oxyalkylene)copolymers, such as hydrophobically modified poly(oxyalkylene)copolymers, polyvinyl pyrrolidone, polyvinyl alcohol, polyvinyl caprolactam, polyvinyl methylether, alkoxylated surfactants, alkoxylated starches, alkoxylated cellulose, alkyl hydroxyalkyl cellulose, silicone-modified polyethers, and poly N-isopropylacrylamide and copolymers thereof. In some embodiments, the polymer is selected from the group consisting of polyether, polyimine, polyalkylene glycol, vinyl polymer, alkoxylated surfactant, polysaccharides, alkoxylated starch, alkoxylated cellulose, alkyl hydroxy alkyl cellulose, polyether-modified silicones, polyacrylamide, polyacrylic acid and copolymer thereof. In some embodiments, the polymer is selected from the group consisting of dipropylene glycol, tripropylene glycol, polyethylene glycol, polypropylene glycol, poly(ethylene glycol-propylene glycol), poly(ethylene glycol-ran-propylene glycol), polyvinyl pyrrolidone, polyvinyl alcohol, polyvinyl caprolactam, polyvinyl methylether, dextran, carboxymethyl dextran, dextran sulfate, hydroxypropyl dextran, starch, carboxymethyl cellulose, poly acrylic acid, hydroxypropyl cellulose, methyl cellulose, ethylhydroxyethylcellulose, maltodextrin, polyethyleneimine, poly N-isopropylacrylamide and copolymers thereof. In some embodiments, the polymer is selected from the group consisting of dipropylene glycol, tripropylene glycol, polyethylene glycol, polypropylene glycol, poly(ethylene glycol-propylene glycol), poly(ethylene glycol-ran-propylene glycol), polyvinyl pyrrolidone, polyvinyl alcohol, polyvinyl caprolactam, polyvinyl methylether and poly N-isopropylacrylamide. In some embodiments, the polymer is selected from the group consisting of polyacrylamide, polyacrylic acid and copolymers thereof. In some embodiments, the polymer is selected from the group consisting of dextran, carboxymethyl dextran, dextran sulfate, hydroxypropyl dextran and starch. In some embodiments, the polymer has an average molecular weight in the range of 200-1,000 Da, 200-35,000 Da, 425-2,000 Da, 400-35,000 Da, 980-12,000 Da, or 3,400-5,000,000 Da. In some embodiments, the polymer comprises ethylene oxide and propylene oxide units, and the polymer has an EO:PO ratio of 90:10 to 10:90.

In one embodiment, the polymer concentration of the first and/or second ATPS is in the range of about 4% to about 84% by weight of the total weight of the aqueous solution (w/w). In various embodiments, the polymer solution is selected from a polymer solution that is about 4% w/w, about 4.5% w/w, about 5% w/w, about 5.5% w/w, about 6% w/w, about 6.5% w/w, about 7% w/w, about 7.5% w/w, about 8% w/w, about 8.5% w/w, about 9% w/w, about 9.5% w/w, about 10% w/w, about 10.5% w/w, about 11% w/w, about 11.5% w/w, about 12% w/w, about 12.5% w/w, about 13% w/w, about 13.5% w/w, about 14% w/w, about 14.5% w/w, about 15% w/w, about 15.5% w/w, about 16% w/w, about 16.5% w/w, about 17% w/w, about 17.5% w/w, about 18% w/w, about 18.5% w/w, about 19% w/w, about 19.5% w/w, about 20% w/w, about 20.5% w/w, about 21% w/w, about 21.5% w/w, about 22% w/w, about 22.5% w/w, about 23% w/w, about 23.5% w/w, about 24% w/w, about 24.5% w/w, about 35% w/w, about 35.5% w/w, about 36% w/w, about 36.5% w/w, about 37% w/w, about 37.5% w/w, about 38% w/w, about 38.5% w/w, about 39% w/w, about 39.5% w/w, about 40% w/w, about 40.5% w/w, about 41% w/w, about 41.5% w/w, about 42% w/w, about 42.5% w/w, about 43% w/w, about 43.5% w/w, about 44% w/w, about 44.5% w/w, about 45% w/w, about 45.5% w/w, about 46% w/w, about 46.5% w/w, about 47% w/w, about 47.5% w/w, about 48% w/w, about 48.5% w/w, about 49% w/w, about 49.5% w/w, about 50% w/w, about 50.5% w/w, about 51% w/w, about 51.5% w/w, about 52% w/w, about 52.5% w/w, about 53% w/w, about 53.5% w/w, about 54% w/w, about 54.5% w/w, about 55% w/w, about 55.5% w/w, about 56% w/w, about 56.5% w/w, about 57% w/w, about 57.5% w/w, about 58% w/w, about 58.5% w/w, about 59% w/w, about 59.5% w/w, about 60% w/w, about 60.5% w/w, about 61% w/w, about 61.5% w/w, about 62% w/w, about 62.5% w/w, about 63% w/w, about 63.5% w/w, about 64% w/w, about 64.5% w/w, about 65% w/w, about 65.5% w/w, about 66% w/w, about 66.5% w/w, about 67% w/w, about 67.5% w/w, about 68% w/w, about 68.5% w/w, about 69% w/w, about 69.5% w/w, about 70% w/w, about 70.5% w/w, about 71% w/w, about 71.5% w/w, about 72% w/w, about 72.5% w/w, about 73% w/w, about 73.5% w/w, about 74% w/w, about 74.5% w/w, about 75% w/w, about 75.5% w/w, about 76% w/w, about 76.5% w/w, about 77% w/w, about 77.5% w/w, about 78% w/w, about 78.5% w/w, about 79% w/w, about 79.5% w/w, about 80% w/w, about 80.5% w/w, about 81% w/w, about 81.5% w/w, about 82% w/w, about 82.5% w/w, about 83% w/w, about 83.5% w/w, and about 84% w/w.

In one embodiment, the first and/or second ATPS comprises a salt and thereby forms a salt solution. In some embodiments, the salt includes, but is not limited to, kosmotropic salts, chaotropic salts, inorganic salts containing cations such as straight or branched trimethyl ammonium, triethyl ammonium, tripropyl ammonium, tributyl ammonium, tetramethyl ammonium, tetraethyl ammonium, tetrapropyl ammonium and tetrabutyl ammonium, and anions such as phosphates, sulphate, nitrate, chloride and hydrogen carbonate. In another embodiment, the salt comprises NaCl, Na₃PO₄, K₃PO₄, Na₂SO₄, potassium citrate, (NH₄)₂SO₄, sodium citrate, sodium acetate and combinations thereof. Other salts, e.g. ammonium acetate, may also be used. In another embodiment, the salt is selected from a magnesium salt, a lithium salt, a sodium salt, a potassium salt, a cesium salt, a zinc salt and an aluminum salt. In some embodiments, the salt is selected from a bromide salt, an iodide salt, a fluoride salt, a carbonate salt, a sulfate salt, a citrate salt, a carboxylate salt, a borate salt, and a phosphate salt. In some embodiments, the salt comprises potassium phosphate. In some embodiments, the salt comprises ammonium sulfate.

In one embodiment, the total salt concentration is in the range of about 0.01% to about 90%. A skilled person in the art will understand that the amount of salt needed to form an aqueous two-phase system will be influenced by molecular weight, concentration and physical status of the polymer.

In various embodiments, the salt concentration is about 1%-80% w/w. In various embodiments, the salt concentration is about 1% w/w, about 1.5% w/w, about 2% w/w, about 2.5% w/w, about 3% w/w, about 3.5% w/w, about 4% w/w, about 4.5% w/w, about 5% w/w, about 5.5% w/w, about 6% w/w, about 6.5% w/w, about 7% w/w, about 7.5% w/w, about 8% w/w, about 8.5% w/w, about 9% w/w, about 9.5% w/w, about 10% w/w, about 10.5% w/w, about 11% w/w, about 11.5% w/w, about 12% w/w, about 12.5% w/w, about 13% w/w, about 13.5% w/w, about 14% w/w, about 14.5% w/w, about 15% w/w, about 15.5% w/w, about 16% w/w, about 16.5% w/w, about 17% w/w, about 17.5% w/w, about 18% w/w, about 18.5% w/w, about 19% w/w, about 19.5% w/w, about 20% w/w, about 20.5% w/w, about 21% w/w, about 21.5% w/w, about 22% w/w, about 22.5% w/w, about 23% w/w, about 23.5% w/w, about 24% w/w, about 24.5% w/w, about 35% w/w, about 35.5% w/w, about 36% w/w, about 36.5% w/w, about 37% w/w, about 37.5% w/w, about 38% w/w, about 38.5% w/w, about 39% w/w, about 39.5% w/w, about 40% w/w, about 40.5% w/w, about 41% w/w, about 41.5% w/w, about 42% w/w, about 42.5% w/w, about 43% w/w, about 43.5% w/w, about 44% w/w, about 44.5% w/w, about 45% w/w, about 45.5% w/w, about 46% w/w, about 46.5% w/w, about 47% w/w, about 47.5% w/w, about 48% w/w, about 48.5% w/w, about 49% w/w, about 49.5% w/w, about 50% w/w, about 50.5% w/w, about 51% w/w, about 51.5% w/w, about 52% w/w, about 52.5% w/w, about 53% w/w, about 53.5% w/w, about 54% w/w, about 54.5% w/w, about 55% w/w, about 55.5% w/w, about 56% w/w, about 56.5% w/w, about 57% w/w, about 57.5% w/w, about 58% w/w, about 58.5% w/w, about 59% w/w, about 59.5% w/w, about 60% w/w, about 60.5% w/w, about 61% w/w, about 61.5% w/w, about 62% w/w, about 62.5% w/w, about 63% w/w, about 63.5% w/w, about 64% w/w, about 64.5% w/w, about 65% w/w, about 65.5% w/w, about 66% w/w, about 66.5% w/w, about 67% w/w, about 67.5% w/w, about 68% w/w, about 68.5% w/w, about 69% w/w, about 69.5% w/w, about 70% w/w, about 70.5% w/w, about 71% w/w, about 71.5% w/w, about 72% w/w, about 72.5% w/w, about 73% w/w, about 73.5% w/w, about 74% w/w, about 74.5% w/w, about 75% w/w, about 75.5% w/w, about 76% w/w, about 76.5% w/w, about 77% w/w, about 77.5% w/w, about 78% w/w, about 78.5% w/w, about 79% w/w, about 79.5% w/w, or about 80% w/w.

In one embodiment, the first and/or second ATPS comprises a surfactant. In some embodiments, possible surfactants that may be employed include, but are not limited to, Triton-X, Triton-114, Igepal CA-630 and Nonidet P-40, anionic surfactants, such as carboxylates, sulphonates, petroleum sulphonates, alkylbenzenesulphonates, naphthalenesulphonates, olefin sulphonates, alkyl sulphates, sulphates, sulphated natural oils & fats, sulphated esters, sulphated alkanolamides, alkylphenols, ethoxylated and sulphated, nonionic surfactants, such as ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, polyoxyethylene fatty acid amides, cationic surfactants, such as quaternary ammonium salts, amines with amide linkages, polyoxyethylene alkyl & alicyclic amines, n,n,n′,n′ tetrakis substituted ethylenediamines, 2-alkyl 1-hydroxethyl 2-imidazolines, and amphoteric surfactants, such as n-coco 3-aminopropionic acid/sodium salt, n-tallow 3-iminodipropionate, disodium salt, n-carboxymethyl n dimethyl n-9 octadecenyl ammonium hydroxide, n-cocoamidethyl n hydroxyethylglycine, and sodium salt.

In one embodiment, the surfactant concentration of the first and/or second ATPS is in the range of about 0.05% w/w to about 10% w/w. In various embodiments, the surfactant concentration is about 0.05% w/w, 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3% w/w, 3.1% w/w, about 3.2% w/w, about 3.3% w/w, about 3.4% w/w, about 3.5% w/w, about 3.6% w/w, about 3.7% w/w, about 3.8% w/w, about 3.9% w/w, about 4% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, about 5% w/w, about 5.1% w/w, about 5.2% w/w, about 5.3% w/w, about 5.4% w/w, about 5.5% w/w, about 5.6% w/w, about 5.7% w/w, about 5.8% w/w, about 5.9% w/w, about 6% w/w, 6.1% w/w, about 6.2% w/w, about 6.3% w/w, about 6.4% w/w, about 6.5% w/w, about 6.6% w/w, about 6.7% w/w, about 6.8% w/w, about 6.9% w/w, about 7% w/w, about 7.1% w/w, about 7.2% w/w, about 7.3% w/w, about 7.4% w/w, about 7.5% w/w, about 7.6% w/w, about 7.7% w/w, about 7.8% w/w, about 7.9% w/w, about 8% w/w, about 8.1% w/w, about 8.2% w/w, about 8.3% w/w, about 8.4% w/w, about 8.5% w/w, about 8.6% w/w, about 8.7% w/w, about 8.8% w/w, about 8.9% w/w, about 9% w/w, 9.1% w/w, about 9.2% w/w, about 9.3% w/w, about 9.4% w/w, about 9.5% w/w, about 9.6% w/w, about 9.7% w/w, about 9.8% w/w, about 9.9% w/w, or about 10% w/w.

In one embodiment, the binding buffer (including the second binding buffer) comprises a chaotropic agent. In some embodiments, possible chaotropic agents that may be employed include, but are not limited to, n-butanol, ethanol, guanidinium chloride, guanidinium thiocyanate, lithium perchlorate, lithium acetate, magnesium chloride, phenol, 2-propanol, sodium dodecyl sulfate, thiourea, and urea.

In one embodiment, the concentration of the chaotropic agent in the binding buffer is in the range of about 0.1 M to 8 M. In various embodiments, the concentration of the chaotropic agent is about 0.1 M, about 0.2 M, about 0.3 M, about 0.4 M, about 0.5 M, about 0.6 M, about 0.7 M, about 0.8 M, about 0.9 M, about 1 M, about 1.1 M, about 1.2 M, about 1.3 M, about 1.4 M, about 1.5 M, about 1.6 M, about 1.7 M, about 1.8 M, about 1.9 M, about 2 M, about 2.1 M, about 2.2 M, about 2.3 M, about 2.4 M, about 2.5 M, about 2.6 M, about 2.7 M, about 2.8 M, about 2.9 M, about 3 M, about 3.1 M, about 3.2 M, about 3.3 M, about 3.4 M, about 3.5 M, about 3.6 M, about 3.7 M, about 3.8 M, about 3.9 M, about 4 M, about 4.1 M, about 4.2 M, about 4.3 M, about 4.4 M, about 4.5 M, about 4.6 M, about 4.7 M, about 4.8 M, about 4.9 M, about 5 M, about 5.1 M, about 5.2 M, about 5.3 M, about 5.4 M, about 5.5 M, about 5.6 M, about 5.7 M, about 5.8 M, about 5.9 M, about 6 M, about 6.1 M, about 6.2 M, about 6.3 M, about 6.4 M, about 6.5 M, about 6.6 M, about 6.7 M, about 6.8 M, about 6.9 M, about 7 M, about 7.1 M, about 7.2 M, about 7.3 M, about 7.4 M, about 7.5 M, about 7.6 M, about 7.7 M, about 7.8 M, about 7.9 M, or about 8 M.

In some embodiments, the fractionation buffer comprises a polymer, a salt, a surfactant, a chaotropic agent or combinations thereof. In some embodiments, possible polymers, salts, surfactants and chaotropic agents that may be employed include, but are not limited to, those that are described above.

In some embodiments, the possible magnetic beads that may be employed include, but are not limited to, those that are listed in Table 1.1 below.

TABLE 1.1
Examples of magnetic beads

| Manufacturer | Bead Name | Specification |
|---|---|---|
| MagQu | MF-SIL-5024 | Silica (SiO₂) |
| MagQu | MF-SIL-5010 | Silica (SiO₂) |
| MagQu | MF-NHH-3000 | Amine (—NH2) |
| MagQu | MF-Dex-3000 | Hydroxyl (—OH) |
| Chemagen | M-PVA 011 | Unmodified |
| Chemagen | M-PVA 012 | Unmodified |
| Chemagen | M-PVA 021 | Highly |
| | | carboxylated |
| Chemagen | M-PVA 022 | Highly |
| | | carboxylated |
| Omega Biotek | Mag-Bind ® Particles CH | Silica (SiO₂) |
| Thermofisher | Dynabeads ™ MyOne ™ | Silica-liked coated |
| | SILANE | |
| Ocean | PureBind T Bead | Silica (SiO₂) |
| Nanotech | | |
| Ocean | PureBind M Bead | Silica (SiO₂) |
| Nanotech | | |

### Embodiment 2

In some embodiments, provided is a method for isolating target nucleic acids below a target size from a sample including nucleic acid components; including the steps of: (a) preparing a sample solution from the sample; (b) contacting a plurality of beads with the sample solution, wherein the nucleic acid components bind to the plurality of beads to form a beads-analyte complex; (c) mixing the beads-analyte complex with a fractionation buffer including at least one chaotropic agent to form a bulk fractionation solution, wherein the target nucleic acids below the target size are released from the beads-analyte complex into the bulk fractionation solution; (d) immobilizing the beads-analyte complex; and (e) separating the bulk fractionation solution including the isolated target nucleic acids below the target size from the immobilized beads-analyte complex.

In some embodiments, the step (a) further includes (a1) adding the sample to a first aqueous two-phase system (ATPS) to form a mixture that partitions into a first target-rich phase and a first target-poor phase, wherein the nucleic acid components are concentrated in the first target-rich phase; and (a2) isolating the first target-rich phase containing the concentrated nucleic acid components, resulting in the sample solution.

In some embodiments, the step (a) further includes the following steps after step (a2): (a3) adding the sample solution in step (a2) to a second ATPS to form a second mixture that partitions into a second target-rich phase and a second target-poor phase, wherein the nucleic acid components are concentrated in the second target-rich phase; and (a4) isolating the second target-rich phase containing the concentrated nucleic acid components to form the sample solution in step (a).

In some embodiments, the plurality of beads and the sample solution of step (a) are mixed with a binding buffer prior to the step (b), wherein the binding buffer includes at least one chaotropic agent.

In some embodiments, the step (e) further includes the steps of: (e1) mixing the bulk fractionation solution with a target binding buffer and a plurality of second beads, such that the plurality of second beads bind the target nucleic acids below the target size to form a second beads-analyte complex, wherein the target binding buffer includes at least one chaotropic agent; and (e2) recovering the target nucleic acids below the target size from the second beads-analyte complex.

In some embodiments, the plurality of beads is magnetic beads, silica-based beads, carboxyl beads, hydroxyl beads, amine-coated beads, or any combination thereof.

In some embodiments, the plurality of second beads is magnetic beads, silica-based beads, carboxyl beads, hydroxyl beads, amine-coated beads, or any combination thereof.

In some embodiments, the plurality of beads is magnetic beads, and the step (b) further includes the steps of: (b1) immobilizing the beads-analyte complex by applying a magnetic field to separate the beads-analyte complex from a bulk supernatant; (b2) removing the bulk supernatant; and (b3) removing the magnetic field and proceeding to step (c).

In some embodiments, the plurality of second beads is magnetic beads, and the target nucleic acids recovery of step (e2) further includes steps of: (i) immobilizing the second beads-analyte complex by applying a first magnetic field to separate the second beads-analyte complex from a first supernatant; (ii) removing the first supernatant; (iii) washing the immobilized second beads-analyte complex with a washing buffer; (iv) discarding the washing buffer; (v) removing the first magnetic field; (vi) mixing the second beads-analyte complex with an elution buffer to form a bulk elution solution, wherein the target nucleic acids below the target size are separated from the magnetic beads in the second beads-analyte complex and released into the bulk elution solution; (vii) immobilizing the magnetic beads by applying a second magnetic field; (viii) collecting the bulk elution solution including the isolated target nucleic acids below the target size.

In some embodiments, provided is a method, further including the step of: (f) subjecting the isolated target nucleic acids to a diagnostic assay for detection, quantification, characterization, or combinations thereof, of the target nucleic acids.

In some embodiments, the at least one chaotropic agent of the fractionation buffer is selected from the group consisting of thiocyanate, isothiocyanate, perchlorate, acetate, trichloroacetate, trifluoroacetate, chloride, and iodide.

In some embodiments, the at least one chaotropic agent of the fractionation buffer is selected from the group consisting of guanidinium hydrochloride (GHCl), guanidinium thiocyanate, guanidinium isothiocyanate (GITC), sodium thiocyanate, sodium iodide, sodium perchlorate, sodium trichloroacetate, sodium trifluoroacetate, lithium perchlorate, lithium acetate, magnesium chloride, phenol, 2-propanol, thiourea, and urea.

In some embodiments, the at least one chaotropic agent has a concentration of around 1.5-8M in the fractionation buffer.

In some embodiments, the at least one chaotropic agent is present at a concentration of around 1.8-3.9M in the fractionation buffer.

In some embodiments, the at least one chaotropic agent is present at a concentration of around 1.8-3.0M in the fractionation buffer.

In some embodiments, the fractionation buffer further includes at least one polymer selected from the group consisting of polyvinyl alcohol, polyethylene glycol, polypropylene glycol, dextran, poly(ethylene glycol-ran-propylene glycol), pluronics, polyvinylpyrolidone, and polyacrylate.

In some embodiments, the at least one polymer is present at a concentration of around 0.1-15% (w/w) in the fractionation buffer.

In some embodiments, the at least one polymer is present at a concentration of around 1.0-5.0% (w/w) in the fractionation buffer.

In some embodiments, the at least one polymer is a polymer having an average molecular weight range from 100 to 35,000 Da.

In some embodiments, the fractionation buffer further includes one or more of a pH buffer, metal chelator, or combination thereof.

Examples of pH buffer includes, but are not limited to, phosphate buffer, acetic acid-sodium acetate buffer, citrate-sodium citrate buffer, citrate-NaOH—HCl buffer, sodium borate buffer, carbonate buffer, HEPES buffer, MOPS buffer, TAE buffer, TBST buffer, Tris-HCl buffer, TE Buffer, and TEN Buffer.

Examples of metal chelator includes, but are not limited to, 2,2′-Bipyridyl, dimercaptopropanol, ethylene diamino tetraacetic acid (EDTA), ethylene glycol tetraacetic acid (EGTA), ionophores nitrilotriacetic acid (NTA), salicylic acid, and triethanolamine (TEA).

In some embodiments, the nucleic acid components and/or the target nucleic acids are DNA, RNA or combinations thereof.

In some embodiments, the nucleic acid components and/or the target nucleic acids are cDNA, plasmid DNA, cell-free DNA (cfDNA), circulating tumor DNA (ctDNA), circulating fetal DNA, micro RNA (miRNA), messenger RNA (mRNA), transfer RNA (tRNA), ribosomal RNA (rRNA), or combinations thereof.

In some embodiments, the first ATPS includes first ATPS components capable of forming the first target-rich phase and the first target-poor phase when the first ATPS components are dissolved in an aqueous solution, wherein the first ATPS components are selected from the group consisting of a polymer, salt, surfactant, and combinations thereof.

In some embodiments, the second ATPS includes second ATPS components capable of forming the second target-rich phase and the second target-poor phase when the second ATPS components are dissolved in an aqueous solution, wherein the second ATPS components are selected from the group consisting of a polymer, salt, surfactant, and combinations thereof.

In some embodiments, said polymer dissolves in the aqueous solution at a concentration of 0.5%-80% (w/v).

In some embodiments, the polymer is selected from the group consisting of polyether, polyimine, polyalkylene glycol, vinyl polymer, alkoxylated surfactant, polysaccharides, alkoxylated starch, alkoxylated cellulose, alkyl hydroxyalkyl cellulose, polyether-modified silicones, polyacrylamide, polyacrylic acid and copolymers thereof. In some embodiments, the polymer is hydrophobically-modified, or silicone-modified.

In some embodiments, the polymer is dipropylene glycol, tripropylene glycol, polyethylene glycol, polypropylene glycol, poly(ethylene glycol-propylene glycol), poly(ethylene glycol-ran-propylene glycol), polyvinyl pyrrolidone, polyvinyl alcohol, polyvinyl caprolactam, polyvinyl methylether, dextran, carboxymethyl dextran, dextran sulfate, hydroxypropyl dextran, starch, carboxymethyl cellulose, polyacrylic acid, hydroxypropyl cellulose, methyl cellulose, ethylhydroxyethylcellulose, maltodextrin, polyethyleneimine, poly N-isopropylacrylamide or copolymers thereof.

In some embodiments, the polymer is dipropylene glycol, tripropylene glycol, polyethylene glycol, polypropylene glycol, poly(ethylene glycol-propylene glycol), poly(ethylene glycol-ran-propylene glycol), polyvinyl pyrrolidone, polyvinyl alcohol, polyvinyl caprolactam, polyvinyl methylether or poly N-isopropylacrylamide.

In some embodiments, the polymer is polyacrylamide, polyacrylic acid or copolymers thereof.

In some embodiments, the polymer is a polyacrylamide, polyacrylic acid or copolymers thereof. In some embodiments, the polymer is dextran, carboxymethyl dextran, dextran sulfate, hydroxypropyl dextran or starch.

In some embodiments, the polymer has an average molecular weight in the range of 200-1,000 Da, 200-35,000 Da, 425-2,000 Da, 400-35,000 Da, 980-12,000 Da, or 3,400-5,000,000 Da. In some embodiments, the polymer comprises ethylene oxide and propylene oxide units. In some embodiments, the polymer has an EO:PO ratio of 90:10 to 10:90.

In some embodiments, the polymer has an average molecular weight in the range of 980-12000 Da or 3,400-5,000,000 Da.

In some embodiments, said polymer has an average molecular weight range from 100 to 10,000 Da.

In some embodiments, said salt is dissolved in the aqueous solution at a concentration of 0.1%-80% (w/w).

In some embodiments, said salt is dissolved in the aqueous solution at a concentration of 0.1%-50% (w/w).

In some embodiments, the salt includes a cation selected from the group consisting of sodium, potassium, calcium, ammonium, lithium, magnesium, aluminium, cesium, barium, straight or branched trimethyl ammonium, triethyl ammonium, tripropyl ammonium, tributyl ammonium, tetramethyl ammonium, tetraethyl ammonium, tetrapropyl ammonium and tetrabutyl ammonium.

In some embodiments, the salt includes an anion selected from the group consisting of phosphate, hydrogen phosphate, dihydrogen phosphate, sulfate, sulfide, sulfite, hydrogen sulfate, carbonate, hydrogen carbonate, acetate, nitrate, nitrite, sulfite, chloride, fluoride, chlorate, perchlorate, chlorite, hypochlorite, bromide, bromate, hypobromite, iodide, iodate, cyanate, thiocyanate, isothiocyanate, oxalate, formate, chromate, dichromate, permanganate, hydroxide, hydride, citrate, borate, and tris.

In some embodiments, the salt is selected from the group consisting of aluminum chloride, aluminum phosphate, aluminum carbonate, magnesium chloride, magnesium phosphate, and magnesium carbonate.

In some embodiments, said salt is selected from the group consisting of KCl, NH₄Cl, Na₃PO₄, K₃PO₄, Na₂SO₄, K₂HPO₄, KH₂PO₄, Na₂HPO₄, NaH₂PO₄, (NH₄)₃PO₄, (NH₄)₂HPO₄, NH₄H₂PO₄, potassium citrate, (NH₄)₂SO₄, sodium citrate, sodium acetate, magnesium acetate, sodium oxalate, sodium borate, and ammonium acetate.

In some embodiments, the salt is selected from the group consisting of (NH₄)₃PO₄, sodium formate, ammonium formate, K₂CO₃, KHCO₃, Na₂CO₃, NaHCO₃, MgSO₄, MgCO₃, CaCO₃, CsOH, Cs₂CO₃, Ba(OH)₂, and BaCO3.

In some embodiments, the salt is selected from the group consisting of NH₄Cl, NH₄OH, tetramethyl ammonium chloride, tetrabutyl ammonium chloride, tetramethyl ammonium hydroxide, and tetrabutyl ammonium hydroxide.

In some embodiments, said surfactant dissolves in the aqueous solution at a concentration of 0.05%-10% (w/w).

In some embodiments, the surfactant is selected from the group consisting of anionic surfactant, nonionic surfactant, cationic surfactant, and amphoteric surfactant; wherein the anionic surfactant is carboxylates, sulphonates, petroleum sulphonates, alkylbenzenesulphonates, naphthalenesulphonates, olefin sulphonates, alkyl sulphates, sulphates, sulphated natural oils, sulphated natural fats, sulphated esters, sulphated alkanolamides, sulphated alkylphenols, ethoxylated alkylphenols, or sodium N-lauroyl sarcosinate (NLS); wherein the nonionic surfactant is ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, or polyoxyethylene fatty acid amides; wherein the cationic surfactant is quaternary ammonium salts, amines with amide linkages, polyoxyethylene alkyl amines, polyoxyethylene alicyclic amines, n,n,n′,n′ tetrakis substituted ethylenediamines, or 2-alkyl 1-hydroxethyl 2-imidazolines; wherein the amphoteric surfactant is n-coco 3-aminopropionic acid or sodium salt thereof, n-tallow 3-iminodipropionate or disodium salt thereof, n-carboxymethyl n dimethyl n-9 octadecenyl ammonium hydroxide, or n-cocoamidethyl n hydroxyethylglycine or sodium salt thereof.

In some embodiments, the surfactant is selected from the group consisting of Triton X-100, Triton X-114, Triton X-45, Tween 20, Igepal CA630, Brij 58, Brij O10, Brij L23, Pluronic L-61, Pluronic F-127, sodium dodecyl sulfate, sodium cholate, sodium deoxycholate, N-lauroyl sarcosine sodium salt, Hexadecyltrimethlammonium bromide, and span 80.

In some embodiments, the at least one chaotropic agent of the binding buffer includes an anion selected from the group consisting of thiocyanate, isothiocyanate, perchlorate, acetate, trichloroacetate, trifluoroacetate, chloride, and iodide.

In some embodiments, the at least one chaotropic agent of the binding buffer is selected from the group consisting of guanidinium hydrochloride (GHCl), guanidinium thiocyanate, guanidinium isothiocyanate (GITC), sodium thiocyanate, sodium iodide, sodium perchlorate, sodium trichloroacetate, sodium trifluroacetate, lithium perchlorate, lithium acetate, magnesium chloride, phenol, 2-propanol, thiourea, and urea.

In some embodiments, the binding buffer includes guanidinium and optionally further includes at least one polymer.

In some embodiments, the at least one chaotropic agent of the target binding buffer includes an anion selected from the group consisting of thiocyanate, isothiocyanate, perchlorate, acetate, trichloroacetate, trifluoroacetate, chloride, and iodide.

In some embodiments, the at least one chaotropic agent of the target binding buffer is selected from the group consisting of guanidinium hydrochloride (GHCl), guanidinium thiocyanate, guanidinium isothiocyanate (GITC), sodium thiocyanate, sodium iodide, sodium perchlorate, sodium trichloroacetate, sodium trifluroacetate, lithium perchlorate, lithium acetate, magnesium chloride, phenol, 2-propanol, thiourea, and urea.

In some embodiments, the target binding buffer includes guanidinium and optionally further includes at least one polymer.

In some embodiments, the sample is blood, plasma, urine, saliva, stool, cerebrospinal fluid (CSF), lymph, serum, sputum, peritoneal fluid, sweat, tears, nasal swab, vaginal swab, endocervical swab, semen, or breast milk.

In some embodiments, the step (a) includes the step of: preparing a DNA library from the sample, resulting in the sample solution.

Some embodiments provide a method of diagnosing or determining the risk of a cancer in a subject comprising the steps of
(i) isolating target nucleic acids from a biological sample from the subject using a method described herein;
(ii) measuring the presence of the target nucleic acids and determining whether the subject has or is at risk of having the cancer.

In some embodiments, the target nucleic acids are cell-free DNA and circulating tumor DNA, whereby the method increases a ratio of circulating tumor DNA:cell-free DNA, and/or variant allele frequency (VAF) in the sample for cancer diagnostic assay.

Some embodiments provide a method of treating a cancer in a subject comprising diagnosing the cancer in the subject by steps (i) and (ii) above, and further comprising the step of treating the subject if the subject is determined to have or is at risk of having the cancer.

Some embodiments provide a method of diagnosing or determining the risk of a genetic disease or disorder in a fetus comprising the steps of
(i) isolating target nucleic acids from a biological sample from the fetus's mother using a method described herein;
(ii) measuring the presence of the target nucleic acids and determining whether the fetus has or is at risk of having the genetic disease or disorder.

In some embodiments, the target nucleic acids are circulating fetal DNA, whereby the method enriches fetal fraction in the sample for non-invasive prenatal testing. For the sake of clarity, fetal fraction refers to the fraction of all DNA circulating in a mother's blood that originates from the fetus.

Some embodiments provide a method of treating a genetic disease or disorder in a fetus comprising diagnosing the genetic disease or disorder in the fetus comprising the steps (i) and (ii) above, and further comprising the step of treating the fetus or the fetus's mother if the fetus is determined to have or is at risk of having the genetic disease or disorder.

Methods for measuring the presence of target nucleic acids and determining the presence, risk, or absence of diseases such as cancers and fetal genetic diseases and disorders are known to one of skill in the art.

In some embodiments, provided is a kit for isolating target nucleic acids below a target size from a sample including nucleic acid components, including: (a) at least one ATPS components selected from the group consisting of a polymer, salt, surfactant, and combinations thereof; (b) a plurality of beads; (c) a fractionation buffer including at least one chaotropic agent selected from the group consisting of thiocyanate, isothiocyanate, perchlorate, acetate, trichloroacetate, trifluoroacetate, chloride, and iodide; and (d) a binding buffer includes at least one chaotropic agent selected from the group consisting of thiocyanate, isothiocyanate, perchlorate, acetate, trichloroacetate, trifluoroacetate, chloride, and iodide.

In some embodiments, the plurality of beads is magnetic beads, silica-based beads, carboxyl beads, hydroxyl beads, amine-coated beads, or any combination thereof.

In some embodiments, the at least one chaotropic agent of the fractionation buffer is selected from the group consisting of guanidinium hydrochloride (GHCl), guanidinium thiocyanate, guanidinium isothiocyanate (GITC), sodium thiocyanate, sodium iodide, sodium perchlorate, sodium trichloroacetate, sodium trifluroacetate, lithium perchlorate, lithium acetate, magnesium chloride, phenol, 2-propanol, thiourea, and urea.

In some embodiments, the at least one chaotropic agent has a concentration of around 1.5-8M in the fractionation buffer.

In some embodiments, the fractionation buffer further includes at least one polymer selected from the group consisting of polyvinyl alcohol, polyethylene glycol, polypropylene glycol, dextran, poly(ethylene glycol-ran-propylene glycol), pluronics, polyvinylpyrolidone, and polyacrylate.

In some embodiments, the at least one polymer is present at a concentration of around 0.1-15% (w/w) in the fractionation buffer.

In some embodiments, the at least one polymer has an average molecular weight range from 100 to 35,000 Da.

In some embodiments, the fractionation buffer further includes one or more of a pH buffer, metal chelator, or combination thereof.

In some embodiments, provided is a method for isolating target nucleic acids below a target size from a sample including nucleic acid components; including the steps of: (a) preparing a sample solution from the sample; (b) contacting the sample solution with a solid phase medium configured to selectively bind the nucleic acid components, such that the nucleic acid components bind to the solid phase medium to form a medium-analyte complex; (c) adding a fractionation buffer including at least one chaotropic agent to the medium-analyte complex to form a bulk fractionation solution, wherein the target nucleic acids below the target size are released from the solid phase medium into the bulk fractionation solution; and (d) separating the bulk fractionation solution including the isolated target nucleic acids below the target size from the solid phase medium.

In some embodiments, the solid phase medium is a solid phase extraction column.

In some embodiments, the solid phase extraction column is a spin column.

In some embodiments, the solid phase medium is a plurality of beads.

In some embodiments, the plurality of beads is magnetic beads, silica-based beads, carboxyl beads, hydroxyl beads, amine-coated beads, or any combination thereof.

In some embodiments, the polymer is at a concentration of 0.5-80% (w/v) of the first ATPS and/or the second ATPS. In some embodiments, the polymer is at a concentration of 0.5-30% (w/v) of the first ATPS and/or the second ATPS. In some embodiments, the polymer is at a concentration of 5-60% (w/v) of the first ATPS and/or the second ATPS. In some embodiments, the polymer is at a concentration of 12-50% (w/v) of the first ATPS and/or the second ATPS.

In some embodiments, the salt is at a concentration of 0.1%-80% (w/v) of the first ATPS and/or the second ATPS. In some embodiments, the salt is at a concentration of 5%-60% (w/v) of the first ATPS and/or the second ATPS. In some embodiments, the salt is at a concentration of 0.1%-50% (w/v) of the first ATPS and/or the second ATPS. In some embodiments, the salt is at a concentration of 0.1%-20% (w/v) of the first ATPS and/or the second ATPS. In some embodiments, the salt is at a concentration of 0.01%-30% (w/v). In some embodiments, the salt is at a concentration of 0.01%-10% (w/v) of the first ATPS and/or the second ATPS.

In some embodiments, the surfactant is at a concentration of 0.1-50% (w/v) of the first ATPS and/or the second ATPS. In some embodiments, the surfactant is at a concentration of 0.01%-10% (w/v) of the first ATPS and/or the second ATPS.

In some embodiments, the first ATPS composition is polymer-salt based, comprising at least one polymer at a concentration of 5-80% (w/v) and at least one salt at a concentration of 0.1-80% (w/v). In some embodiments, the first ATPS composition comprises at least one polymer at a concentration of 5-60% (w/v) and at least one salt at a concentration of 0.5-50% (w/v). In some embodiments, the first ATPS composition comprises at least one polymer at a concentration of 12-50% (w/v) and at least one salt a concentration of 0.1-20% (w/v). In some embodiments, the first ATPS composition further comprises at least one surfactant at a concentration of 0.01%-10% (w/v).

In some embodiments, the second ATPS composition comprises at least one polymer at a concentration of 0.5-30% (w/v) and at least one salt at a concentration of 5-60% (w/v). In some embodiments, the second ATPS composition comprises at least one polymer at a concentration of 1-6% (w/v) and at least one salt at a concentration of 10-50% (w/v). In some embodiments, the second ATPS composition further comprises at least one surfactant at a concentration of 0.01%-10% (w/v).

In some embodiments, the first ATPS composition is polymer-salt based, comprising at least one polymer at a concentration of 0.5-30% (w/v) and at least one salt at a concentration of 5-60% (w/v). In some embodiments, the first ATPS composition comprises at least one polymer at a concentration of 1-6% (w/v) and at least one salt at a concentration of 10-50% (w/v). In some embodiments, the first ATPS composition further comprises at least one surfactant at a concentration of 0.01%-10% (w/v).

In some embodiments, the second ATPS composition comprises at least one polymer at a concentration of 5-80% (w/v) and at least one salt at a concentration of 0.1-80% (w/v). In some embodiments, the second ATPS composition comprises at least one polymer at a concentration of 5-60% (w/v) and at least one salt at a concentration of 0.5-50% (w/v). In some embodiments, the second ATPS composition comprises at least one polymer at a concentration of 12-50% (w/v) and at least one salt at a concentration of 0.1-20% (w/v). In some embodiments, the second ATPS composition further comprises at least one surfactant at a concentration of 0.01%-10% (w/v).

In some embodiments, the first ATPS composition is polymer-polymer based, comprising at least one polymer at a concentration of 0.2-50% (w/v). In some embodiments, the first ATPS composition further comprises at least one salt at a concentration of 0.01%-10% (w/v). In some embodiments, the first ATPS composition further comprises at least one surfactant at a concentration of 0.01%-10% (w/v).
In some embodiments, the first ATPS composition is surfactant based, comprising at least one surfactant at a concentration of 0.1-50% (w/v). In some embodiments, the first ATPS composition further comprises at least one salt at a concentration of 0.01%-30% (w/v).

Although the description referred to particular embodiments, the disclosure should not be construed as limited to the embodiments set forth herein.

### EXAMPLES

Provided herein are examples that describe in more detail certain embodiments of the present disclosure. The examples provided herein are merely for illustrative purposes and are not meant to limit the scope of the invention in any way. All references given below and elsewhere in the present application are hereby included by reference.

### Example 1: Protocol for Concentrating DNA without Fractionation Buffer

Below is an example method of how to concentrate and isolate a target analyte according to the present disclosure. In this example, the target analyte is DNA. Protocol steps are performed as follows:
1. A desired volume of treated biological sample (e.g., blood plasma) (e.g. 2-3 mL) is added to the first ATPS (Solution B) to form Solution B′. Treatment methods for the biological sample include, but are not limited to, lysing to form a sample lysate.
2. Solution B′ is vortexed thoroughly (e.g., for about 10 seconds) until homogenous, and then centrifuged for 6 min at 2,300×g.
3. The bottom phase of Solution B′ is transferred to the second ATPS (Solution C) to form Solution C′.
4. Solution C′ is vortexed thoroughly until homogenous for 10 seconds, and then centrifuged for 1 min at 7,000×g.
5. 800 uL of a binding buffer (e.g., Binding Buffer BB1, BB2, or BB3) is added into a new 2 mL microcentrifuge tube.
6. The top phase of Solution C′ containing the concentrated target analyte is transferred to the microcentrifuge tube from Step 5.
7. The provided magnetic beads (e.g. the magnetic beads selected from Table 1, 12 μL) are vortexed before use, and then added into the microcentrifuge tube from Step 6, such that the magnetic beads bind the target analyte to form a beads-analyte complex.
8. The microcentrifuge tube is incubated with tilt rotation for 5 minutes, and then placed on a magnetic stand for 2 minute to immobilize the beads-analyte complex at the tube wall.
9. The supernatant from the microcentrifuge tube is pipetted and discarded without disturbing the beads-analyte complex. The microcentrifuge tube is then removed from the magnetic stand.
10. 800 uL of a binding buffer (e.g., Binding Buffer BB1, BB2, or BB3) is added into the microcentrifuge tube. The microcentrifuge tube is vortexed for 20 seconds, and then placed on the magnetic stand for 2 minute to immobilize the beads-analyte complex at tube wall.
11. The supernatant from the microcentrifuge tube is pipetted and discarded without disturbing the beads-analyte complex.
12. Suitable Washing Buffer (e.g. 800 uL) known to one skilled in the art is added into the microcentrifuge tube, which is then rotated on the magnetic stand, 120 degree each time, rotating a total of 720 degrees. After rotation, the supernatant from the microcentrifuge tube is pipetted and discarded without disturbing the beads-analyte complex.
13. Step 12 is repeated at least one time.
14. The microcentrifuge tube is then briefly spun with the hinge facing outwards to collect any remaining washing buffer in the tube.
15. The microcentrifuge tube is placed back on the magnetic stand for 1 minute to immobilize the beads-analyte complex at tube wall.
16. All supernatant is discarded carefully (e.g., using 10 μl pipette tips) without disturbing the beads-analyte complex.
17. The tube cap is opened and the beads-analyte complex is allowed to dry for 7 minutes on the magnetic rack.
18. The microcentrifuge tube is then removed from the magnetic stand after drying.
19. Suitable Elution Buffer (e.g. 40 μL) known to one skilled in the art is added directly to the beads-analyte complex (in the microcentrifuge tube).
20. The beads-analyte complex is resuspended by continuous stirring using a pipette and then pipetted up-and-down for 5 times.
21. The microcentrifuge tube is vortexed mildly (e.g., for 15 seconds).
22. The microcentrifuge tube is incubated at room temperature for 3 minutes.
23. The microcentrifuge tube is placed on the magnetic stand for 1 minute.
24. The supernatant containing the purified target analyte is collected into a clean Maximum Recovery tube carefully without disturbing the magnetic beads.
25. The purified target analyte is ready for immediate use or long-term storage at −20° C. or below.

### Example 2: Protocol for Concentrating DNA with Fractionation Buffer

Below is another example method of how to concentrate and isolate a target analyte according to the present disclosure. In this example, the target analyte is DNA.

Protocol steps are performed as follows:
1. A desired volume of treated biological sample (e.g., blood plasma) (e.g 2-3 mL) is added to the first ATPS (Solution B) to form Solution B′. Treatment methods for the biological sample include, but are not limited to, lysing to form a sample lysate.
2. Solution B′ is vortexed thoroughly until homogenous for 10 seconds, and then centrifuged for 6 min at 2,300×g.
3. The bottom phase of Solution B′ is transferred to the second ATPS (Solution C) to form Solution C′.
4. Solution C′ is vortexed thoroughly until homogenous for 10 seconds, and then centrifuged for 1 min at 7,000×g.
5. 800 uL of binding buffer (e.g., Binding Buffer BB1, BB2, or BB3) is added into a new 2 mL microcentrifuge tube.
6. The top phase of Solution C′ containing the concentrated target analyte is transferred to the tube filled with binding buffer from Step 5.
7. The provided magnetic beads (e.g. the magnetic beads selected from Table 1, 12 μL) are vortexed before use, and then added into the microcentrifuge tube from Step 6, such that the magnetic beads bind the target analyte to form a beads-analyte complex.
8. The microcentrifuge tube is incubated with tilt rotation for 5 minutes, and then placed on the magnetic stand for 2 minute to immobilize the beads-analyte complex at tube wall.
9. The supernatant from the microcentrifuge tube is pipetted and discarded without disturbing the beads-analyte complex. The microcentrifuge tube is then removed from the magnetic stand.
10. 300 uL Fractionation Buffer (e.g. Fractionation Buffer F1, F2 or F3) is added into the microcentrifuge tube. The microcentrifuge tube is vortexed for 20 seconds, incubated with tilt rotation for 5 minutes, and then placed on the magnetic stand for 2 minute to immobilize the beads-analyte complex at tube wall, such that the target analyte below a target size is released from the beads-analyte complex into the supernatant.
11. 600 uL of a second Binding Buffer (e.g., Binding Buffer BB1, BB2, or BB3) is added into a new 2 mL microcentrifuge tube.
12. The supernatant from Step 10 are transferred into the tube filled with the second Binding Buffer from Step 11.
13. The mixture is pipetted up and down to make sure all supernatant was transferred and mixed well with the second Binding Buffer.
14. The provided magnetic beads (e.g. the magnetic beads selected from Table 1, 6 μL) are vortexed before use, and then added into the microcentrifuge tube from Step 13, such that the magnetic beads bind the target analyte below the target size to form a second beads-analyte complex.
15. The microcentrifuge tube is incubated with tilt rotation for 5 minutes, and then placed on the magnetic stand for 4 minute to immobilize the second beads-analyte complex at tube wall.
16. The supernatant from the microcentrifuge tube is pipetted and discarded without disturbing the second beads-analyte complex.
17. Suitable Washing Buffer (e.g. 800 uL) known to one skilled in the art is added into the microcentrifuge tube, which is then rotated on magnetic stand, 120 degree each time, rotating a total of 720 degrees. After rotation, the supernatant from the microcentrifuge tube is pipetted and discarded without disturbing the second beads-analyte complex.
18. Step 17 is repeated.
19. The tube cap is opened and the beads are allowed to dry for 15 minutes on the magnetic rack.
20. The tube is removed from the magnetic stand after drying.
21. Suitable Elution Buffer (e.g. 40 μL) known to one skilled in the art is added directly to the second beads-analyte complex (in the microcentrifuge tube).
22. The second beads-analyte complex is resuspended by continuous stirring using pipette, then pipetted up-and-down for 5 times.
23. The microcentrifuge tubes are vortexed mildly for 10 seconds.
24. The microcentrifuge tube is incubated at room temperature for 3 minutes.
25. The microcentrifuge tube is placed on the magnetic stand for 1 minute.
26. The supernatant containing the purified target analyte below the target size is collected into a clean Maximum Recovery tube carefully without disturbing the magnetic beads.
27. The purified target analyte below the target size is ready for immediate use or long-term storage at −20° C. or below.

### Example 3: Evaluation of the Performance of the Example Methods

Performance of the presently disclosed methods and kits below can be evaluated following the steps below:
(i) Several Magnetic Bead extraction kits components are prepared by varying the following components:
   a. Solution B
      i. Polymer
      ii. Salt
      iii. Surfactant
   b. Solution C
      i. Polymer
      ii. Salt
   c. Magnetic Beads
   d. Binding Buffer
      i. Chaotropic agent
      ii. Polymer
   e. Fractionation Buffer
      i. Chaotropic agent
      ii. Polymer
(ii) Sample solutions are made to evaluate and spike in known quantities of DNA target.
(iii) Extraction are made using variations of Magnetic Bead extraction kits prepared in Step 1 above as well as industry standard extraction kits using their specified procedures.
(iv) Target DNA are quantified using standard qPCR or ddPCR procedures.

TABLE 2
In various example embodiments, Solution B, Solution C, the Binding
Buffer, and Fractionation Buffer are selected from the Examples
shown below in a variety of different combinations.

| | | | | | Chaotropic |
|---|---|---|---|---|---|
| Reagent | Example | Polymer | Salt | Surfactant | Agent |
| Solution B | B1 | Polyvinyl | Sodium | None | None |
| (first ATPS) | | alcohol 55- | Sulfate 12- | | |
| | | 63% v/v | 15% w/v | | |
| | B2 | Polyethylene | Phosphate | Triton 0.05- | None |
| | | Oxide (POE) | Salt 28-37% | 0.4% v/v | |
| | | 55-65% v/v | w/v | | |
| | B3 | PPG 78-84% | Potassium | Igepal 0.5- | None |
| | | v/v | citrate 19- | 1.8% v/v | |
| | | | 23% w/v | | |
| | B4 | Dextran 42- | Magnesium | Anionic | None |
| | | 57% w/v | Salt 8-12% | Surfactant | |
| | | | w/v | 2-5% v/v | |
| Solution C | C1 | Polyvinyl | Sodium | None | None |
| (second | | alcohol 12- | Sulfate 32- | | |
| ATPS) | | 19% v/v | 55% w/v | | |
| | C2 | PPG 4-20% | Potassium | Igepal 4.5- | None |
| | | v/v | citrate 29- | 9.8% v/v | |
| | | | 43% w/v | | |
| | C3 | Dextran 15- | Magnesium | Anionic | None |
| | | 28% w/v | Salt 20-31% | Surfactant | |
| | | | w/v | 2-5% v/v | |
| | C4 | POE 18-34% | Phosphate | None | None |
| | | v/v | Salt 67-80% | | |
| | | | w/v | | |
| Binding | BB1 | None | None | None | 2.5-6M |
| Buffer | | | | | Guanidinium |
| | | | | | Chloride |
| | BB2 | None | None | None | 3-8M |
| | | | | | Magnesium |
| | | | | | Chloride |
| | BB3 | None | None | None | 4-7M |
| | | | | | Guanidinium |
| | | | | | Thiocyanate |
| Fractionation | F1 | POE 1-5% | None | None | 2-5M |
| Buffer | | v/v | | | Guanidinium |
| | | | | | Thiocyanate |
| | F2 | PPG 78-84% | None | None | 2.5-6M |
| | | v/v | | | Guanidinium |
| | | | | | Chloride |
| | F3 | Dextran 42- | Sodium | Triton 0.05- | 3-8M |
| | | 57% w/v | Sulfate 12- | 0.4% v/v | Magnesium |
| | | | 15% w/v | | Chloride |

### Example 4a: Direct Fractionation and Reverse Fractionation

In some embodiments, provided herein are two different protocols/methods using fractionation buffer to separate target nucleic acids such as DNA into larger and smaller fragments according to their sizes, namely direct fractionation and reverse fractionation.

Direct Fractionation

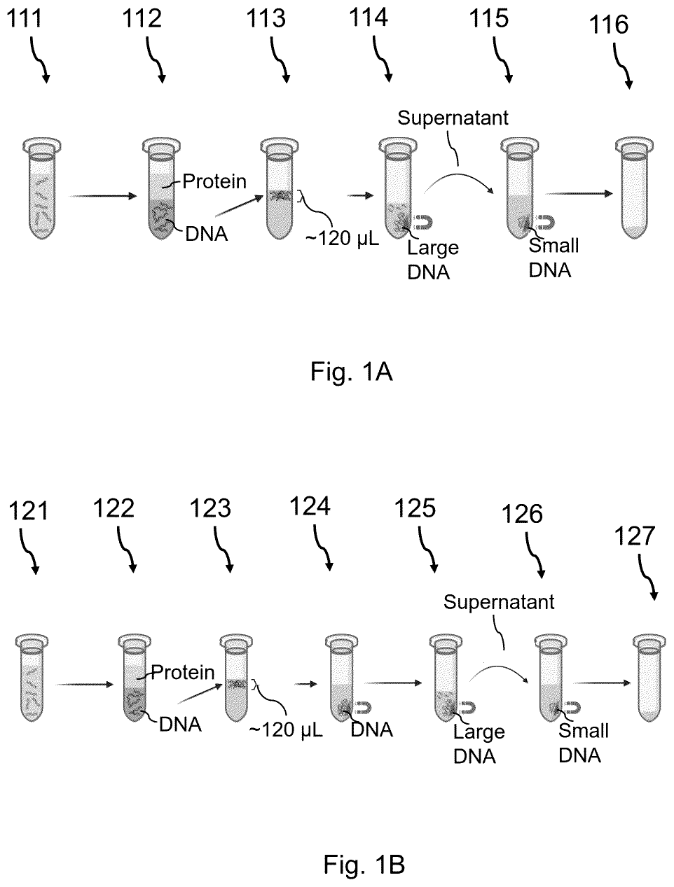

Referring now to FIG. 1A, which shows a general workflow of direct fractionation protocol according to one example embodiment. In this embodiment, in the first optional lysis step 111, suitable lysis buffer is added into the sample (e.g. blood, plasma, serum, cerebrospinal fluid, urine, saliva, fecal matter, tear, sputum, nasopharyngeal mucus, vaginal discharge or penile discharge) for lysing the cells in the sample and releasing the biomolecules into the lysis buffer solution. The lysed sample undergoes two sequential aqueous two-phase systems (ATPSs) to isolate and concentrate DNA. In step 112, the lysed sample is mixed with a first ATPS. The mixture is centrifuged such that it is separated into a top phase and bottom phase. In step 113, all bottom phase where DNA was likely to partitioned to (also referred to as “first target-rich phase”) is transferred into a second ATPS (2^{nd} ATPS) and thoroughly mixed. The mixture is centrifuged such that it is separated into a top phase and a bottom phase. In step 114, the top phase from second ATPS which target DNA would have partitioned to (also referred to as “second target-rich phase”) is extracted into an empty microcentrifuge tube. Direct fractionation buffer (such as those as described in Examples 5-6 below) is added into the tube and mixed well with the top phase from 2^{nd} ATPS as well as magnetic beads that are also added into the tube, such that the magnetic beads only bind the larger DNA fragments. The mixture is incubated for a certain time and is then spun down and placed on a magnetic rack to immobilize the beads at tube wall. Large DNA fragments were bound to the beads while smaller DNA fragments remained in supernatant, which is then extracted and transferred into a new microcentrifuge tube without disturbing the beads (see step 115). Further in step 115, binding buffer (for example, binding buffer A as described in Example 5 below) is added into the extracted supernatant. Magnetic beads are then added into the supernatant-binding buffer mixture and incubated for certain time to bind the smaller DNA fragments. The tube is then spun down and placed on a magnetic rack to immobilize the beads-analyte complex. Supernatant is discarded without disturbing the beads-analyte complex. In step 116, the beads-analyte complex can be further purified by several washing steps (optional) using suitable washing buffer. The beads-analyte complex is then resuspended in suitable elution buffer, mixed well, and then placed on the magnetic rack to immobilize the magnetic beads. The supernatant comprising the purified DNA sample is collected for further use.

Reverse Fractionation

Referring now to FIG. 1B, which shows a general workflow of reverse fractionation protocol according to one example embodiment. In this embodiment, steps 121, 122 and 123 are the same or similar to steps 111, 112 and 113 respectively as described in direct fractionation protocol above. In step 124, the top phase from second ATPS which target DNA would have partitioned to (also referred to as “second target-rich phase”) is extracted into a microcentrifuge tube filled with binding buffer (such as binding buffer A as described in Example 5 below). Magnetic beads are added into the mixture and incubated for certain time. The tube is then spun down and placed on a magnetic rack to immobilize the beads at tube wall. DNA of all sizes should be bound to magnetic beads while proteins and contaminants remained in supernatant, which is discarded. In step 125, reverse fractionation buffer (such as those as described in Examples 5-7 below) is then added to the beads and mixed well. After incubated for certain time, the tube is then spun down and placed on magnetic rack. Large DNA fragments are bound to the beads while smaller DNA fragments remained in supernatant, which is then extracted and transferred into a new microcentrifuge tube without disturbing the beads (see step 126). Further in step 126, target binding buffer (for example, binding buffer B in Example 5 below) and magnetic beads are added into the supernatant to capture fractionated DNA fragments into the beads. The mixture is incubated for certain time and spun down and put on magnetic rack to immobilize the beads-analyte complex. The supernatant is pipetted and discarded. In step 127, the beads-analyte complex can be further purified by several washing steps (optional) using suitable washing buffer. The beads-analyte complex is then resuspended in suitable elution buffer, mixed well, and then placed on the magnetic rack to immobilize the magnetic beads. The supernatant comprising the purified DNA sample is collected for further use.

In some embodiments, the possible magnetic beads that may be employed include, but are not limited to, those that are listed in Table 1.2 below.

TABLE 1.2
Examples of magnetic beads

| Manufacturer | Bead Name | Specification |
|---|---|---|
| G-Biosciences | 786-915 | Silica (SiO₂) |
| Biochain | L5011001 | Silica (SiO₂) |
| MagQu | MF-SIL-5024 | Silica (SiO₂) |
| MagQu | MF-SIL-5010 | Silica (SiO₂) |
| G-Biosciences | 786-915 | Silica (SiO₂) |
| Biochain | L5011001 | Silica (SiO₂) |
| Luna nanotech | NMG-101 | Silica (SiO₂) |
| Cambrian bioworks | CBWD001 | Silica (SiO₂) |
| Bioclone | FF-103 | Silica (SiO₂) |
| Chemagen | M-PVA 011 | Unmodified |
| Chemagen | M-PVA 012 | Unmodified |
| Chemagen | M-PVA 021 | Highly carboxylated |
| Chemagen | M-PVA 022 | Highly carboxylated |
| Omega Biotek | Mag-Bind ® Particles CH | Silica (SiO₂) |
| Thermofisher | Dynabeads ™ MyOne ™ | Silica-liked coated |
| | SILANE | |
| Ocean Nanotech | PureBind T Bead | Silica (SiO₂) |
| Ocean Nanotech | PureBind M Bead | Silica (SiO₂) |
| MagQu | MF-NHH-3000 | Amine (—NH2) |
| MagQu | MF-Dex-3000 | Hydroxyl (—OH) |
| Avanbio | fe10002-cf | Carboxyl |
| Avanbio | fe03001 | Carboxyl |

### Example 4b: Calculation of DNA Cutoff Value

In some embodiments, the DNA cutoff value of a given sample is calculated by the method as described below.

A sample with DNA ladder is extracted using size fractionation procedure according to any of the method as described in the present disclosure. To evaluate the DNA cutoff value, the extracted DNA sample and a positive control condition (representing 100% recovery condition of all sizes DNA) are analyzed using Agilent Bioanalyzer High Sensitivity DNA Kit (cat #5067-4626). The concentrations of each peak are measured by the Agilent 2100 analysis software and exported to a spreadsheet. First, for the positive control condition, the concentration of all DNA fragments greater than 100 bp is divided by the concentration of the 100 bp DNA fragment to calculate the Ratio (Conc/Conc a) (as shown in Table I). This 100 bp DNA size fragment (also referred to as “non-size selected internal 100 bp control”) was chosen because it is not impacted by the size fractionation and therefore serves as a normalization value for a 100% recovery condition within each experiment.

TABLE I
results for positive control condition

| | Positive Control | | |
|---|---|---|---|
| | | Conc. | Ratio |
| | Size [bp] | [pg/μl] | (Conc/Conc_a) |
| a | 104 | 279.98 | 4.978307255 |
| b | 124 | 74.77 | 0.26705479 |
| c | 143 | 89.14 | 0.318379884 |
| d | 152 | 88.74 | 0.316951211 |
| e | 162 | 93.61 | 0.33434531 |
| f | 182 | 85.18 | 0.304236017 |
| g | 201 | 217.83 | 0.778019859 |
| h | 251 | 143.6 | 0.512893778 |
| I | 298 | 177.79 | 0.635009644 |
| j | 354 | 47.8 | 0.17072648 |
| k | 406 | 73.43 | 0.262268733 |
| l | 466 | 83.72 | 0.299021359 |
| m | 517 | 90.3 | 0.322523037 |

The same ratio calculation is performed for the extracted DNA sample (referred to as ““Example A”), as shown in Table II. Then the Recovery % is calculated by dividing the “Ratio” from “Example A” in Table II by the “Ratio” from “Positive Control” in Table I. Values greater than 100% can occur as a result of bioanalyzer artifacts and fluctuations to the baseline signal. All values greater than 100% are assumed to be 100% recovery. The bp size which gives 70% recovery compared to the non-size selected internal 100 bp control is estimated by finding the two recovery % values that straddle 70% and the perform a linear regression between those two points. The resulting value is regarded as the DNA cutoff value of the example.

TABLE II
results for Example A

| | Example A | | Ratio | Recovery | DNA cutoff |
|---|---|---|---|---|---|
| | Size [bp] | Conc. [pg/μl] | (Conc/Conc_a) | % | value |
| a | 105 | 120.62 | 4.782712133 | 96.07% | |
| b | 125 | 33.83 | 0.280467584 | 105.02% | |
| c | 144 | 40.42 | 0.335101973 | 105.25% | |
| d | 153 | 39.67 | 0.328884099 | 103.76% | |
| e | 163 | 45.48 | 0.377051899 | 112.77% | |
| f | 183 | 47.46 | 0.393467087 | 129.33% | |
| g | 202 | 82.88 | 0.687116564 | 88.32% | 70% cutoff |
| h | 253 | 34.91 | 0.289421323 | 56.43% | estimation |
| | | | | | 229 bp |
| i | 300 | 19.67 | 0.163074117 | 25.68% | |

### Example 5

The following study was conducted to compare the stability and robustness of direct fractionation and reverse fractionation protocols for extraction of DNA from plasma with different volumes of sample, in particular different volumes of the top phase of the 2^{nd} ATPS being added to the fractionation step. The ability of the direct fractionation and reverse fractionation protocols to accommodate sample-to-sample variability was assessed.

Materials and Methods

Plasma Cell Lysis

Each 2 mL of plasma was spiked with 100 fg of 145 bp dsDNA oligos, 80 ng 20 bp ladder (Jena Bioscience, Cat #M212) and 40 ng 50 bp ladder (Jena Bioscience, Cat #M-213). 160 μL of suitable lysis buffer and 60 μL of Proteinase K (28.5 mg/mL) was added into each 2 mL spiked plasma. The mixture was vortexed thoroughly then lysed for 15 minutes at a pre-heated 60° C. heat block.

Two-Phase System

Lysed plasma samples undergone two sequential aqueous two-phase systems (ATPSs) to isolate and concentrate DNA. 2.22 mL of lysed sample was transferred to the first ATPS (polymer, salts and/or surfactant) and vortex mixed. The mixture was centrifuged at 2,300 rcf for 6 minutes. All salt-rich bottom phase (˜1 mL) where DNA was likely to partitioned to (also referred to as “first target-rich phase”), was transferred into a second ATPS (polymer, salts and/or surfactant) and thoroughly mixed. The mixture was then centrifuged at 7,000×g for 1 minute. Top phase (150 μL) which target DNA would have partitioned to (also referred to as “second target-rich phase”) was carefully extracted into an empty microcentrifuge tube for further purification. Three replicates were performed, with different top phase volumes of second ATPS (100, 150 and 180 μL) extracted. One sample set with each top phase volume would go through direct fractionation while another set would go through reverse fractionation protocol.

Direct Fractionation

Two direct fractionation buffer formulas which have different expected DNA cutoff sizes were used for each top phase volume of ATPS: (i) Fractionation Buffer D1 for a smaller (around 150 bp) expected cutoff size, i.e. 3-7M guanidinium, 0.1-15% (w/v) of polymer, 0.01M-0.5M pH buffer and 0.01M-0.5M metal chelator; and (ii) Fractionation Buffer D2 for a larger (around 300 bp) expected cutoff size, i.e. 3-7M guanidinium, 0.1-15% (w/v) of polymer, 0.01M-0.5M pH buffer and 0.01M-0.5M metal chelator.

For each sample, the top phase from second ATPS was transferred into an empty microcentrifuge tube. 80 μL of direct fractionation buffer (Fractionation Buffer D1 or D2) was added into the tube and mixed well with 12 μL of magnetic beads that were also added into the tube. Magnetic beads (Cat #MF-SIL-5024) were purchased from MagQu Co. Ltd. The mixture was incubated with tilt rotation for 5 minutes. The tubes were then briefly spun down and placed on a magnetic rack for 2 minutes to immobilize the beads at tube wall. Large DNA fragments were bound to the beads while smaller DNA fragments remained in supernatant, which was then extracted and transferred into a new microcentrifuge tube without disturbing the beads. 600 μL of binding buffer A (3-7M guanidinium and 0.1-15% (w/v) of polymer) which provided the necessary chaotropic salts for salt bridge formation between DNA and solid phase magnetic beads, was added into the extracted supernatant. 6 μL magnetic beads were then added into the supernatant-binding buffer mixture and incubated for another 5 minutes with tilt rotation. The tubes were then spun down and placed on a magnetic rack for 4 minutes. Supernatant was discarded without disturbing the beads-analyte complex. 600 μL of binding buffer A was then again added into beads-analyte complex and the tube was rotated on the rack for 720° in total. Supernatant was discarded. The beads-analyte complex was then proceeded to further purification.

Reverse Fractionation

Two reverse fractionation buffer formulas have been applied to each top phase volume targeting different expected DNA cutoff size: (i) Fractionation Buffer R1 for a smaller (around 150 bp) expected cutoff size, i.e. 3-7M guanidinium, 0.1-15% (w/v) of polymer, 0.01M-0.5M pH buffer and 0.01M-0.5M metal chelator; and (ii) Fractionation Buffer R2 for a larger (around 300 bp) expected cutoff size, i.e. 0.5-5.0M guanidinium, 0.01M-0.5M pH buffer and 0.01M-0.5M metal chelator.

Each top phase volume from second ATPS was transferred into a microcentrifuge tube filled with 800 μL of binding buffer A (3-7M guanidinium and 0.1-15% (w/v) of polymer). 12 μL magnetic beads were added into the mixture and was incubated with tilt rotation for 5 minutes. The tubes were spun down and placed on magnetic rack for 2 minutes to immobilize the beads at tube wall. DNA of all sizes should be bound to magnetic beads while proteins and contaminants remained in supernatant, which was discarded. 300 μL of reverse fractionation buffer (Fractionation Buffer R1 or R2) was then added to the beads. After vortex mixing, the mixture was incubated for 5 minutes on rotator. The tubes were spun down and placed on magnetic rack for another 2 minutes. Larger DNA fragments were bound to the beads while smaller DNA fragments remained in supernatant. The supernatant was then extracted and transferred to a new microcentrifuge tube. 600 μL binding buffer B (3-7M guanidinium and 0.1-15% (w/v) of polymer, also referred to as “target binding buffer” in some embodiments) and 6 μL magnetic beads were added into the supernatant to capture fractionated DNA fragments into the beads. The mixture was incubated on rotator for another 5 minutes and spun down and put on magnetic rack for 4 minutes afterwards. The supernatant was again pipetted and discarded. The beads-analyte complex was then proceeded to further purification.

Purification of DNA

800 μL of washing buffer (70% ethanol, 0.001 M EDTA, 0.01 M Tris-HCl) was added to each processed sample that went through the direct fractionation or reverse fractionation protocol as described above, and the tube was rotated on the magnetic rack for 720° in total. The supernatant was discarded. The washing steps were performed twice. The tubes were briefly spun down using bench-top microcentrifuge with the hinge facing outwards to collect any remaining washing buffer. The beads-analyte complex was then dried for 7 minutes on magnetic stand with cap opened. The beads-analyte complex was resuspended in 40 μL of elution buffer (0.01 M Tris-HCl, 0.001 M EDTA) by continuous pipette mixing, followed by mild vortex. The tube was then placed on the magnetic rack for 1 minute to immobilize the magnetic beads. The supernatant comprising the purified DNA sample was collected carefully into a DNA lo-bind tube for detection without disturbing the magnetic beads.

Detection of DNA

Recovery of different sizes of DNA oligos in extracted samples were quantified by electrophoresis. The gel mixture was prepared by Agilent™ High Sensitivity DNA Kit (Agilent, 5067-4626). 9 μL of gel-dye mix was distributed into each well on the microfluidic chip and 1 μL of purified DNA sample was added. Electrophoresis on a chip was performed by Agilent™ 2100 Bioanalyzer. The fluorescence signals of the reaction were collected and analyzed using the software Agilent™ Technologies 2100 Expert. The actual DNA cutoff value is estimated by the calculation method as described in Example 4b, i.e. calculating the value of base pair of the purified DNA sample in which the DNA recovery % is 70%.

The conditions of sample sets and compositions of the fractionation buffer formulas (for both direct and reverse fractionation) used in this study are summarized in Table 3 below.

TABLE 3
conditions of sample sets and compositions of the fractionation buffer formulas

| | Top | | | |
|---|---|---|---|---|
| | phase | Fractionation buffer | Fractionation | Expected |
| Condition | volume | formula | Method | Cutoff |
| 1 | 100 uL | Fractionation Buffer D1: 3- | Direct | 150 bp |
| 2 | 150 uL | 7M guanidinium, 0.1-15% | Fractionation | |
| 3 | 180 uL | (w/v) of polymer, 0.01M- | | |
| | | 0.5M pH buffer and 0.01M- | | |
| | | 0.5M metal chelator | | |
| 4 | 100 uL | Fractionation Buffer R1: 1- | Reverse | |
| | | 5M guanidinium, 0.1-15% | Fractionation | |
| 5 | 150 uL | (w/v) of polymer, 0.01M- | | |
| | | 0.5M pH buffer and 0.01M- | | |
| 6 | 180 uL | 0.5M metal chelator | | |
| 7 | 100 uL | Fractionation Buffer D2: | Direct | 300 bp |
| | | 1-5M guanidinium, 0.1-15% | Fractionation | |
| 8 | 150 uL | (w/v) of polymer, 0.01M- | | |
| | | 0.5M pH buffer and 0.01M- | | |
| 9 | 180 uL | 0.5M metal chelator | | |
| 10 | 100 uL | Fractionation Buffer R2: 0.5- | Reverse | |
| | | 5.0M guanidinium, 0.01M- | Fractionation | |
| 11 | 150 uL | 0.5M pH buffer and 0.01M- | | |
| 12 | 180 uL | 0.5M metal chelator | | |

Results

Recovery of DNA

The difference in volume of samples, for example the volume of top phase extracted in the 2^{nd} ATPS which is directly used for the subsequent fractionation (direct or reverse fractionation) protocol, may affect the efficiency of DNA fractionation. To address such differences, various top phase volumes of the 2^{nd} ATPS (100, 150 and 180 μL) were used in the fractionation protocol of both direct and reverse fractionation to investigate the stability and robustness of the two types of fractionations.

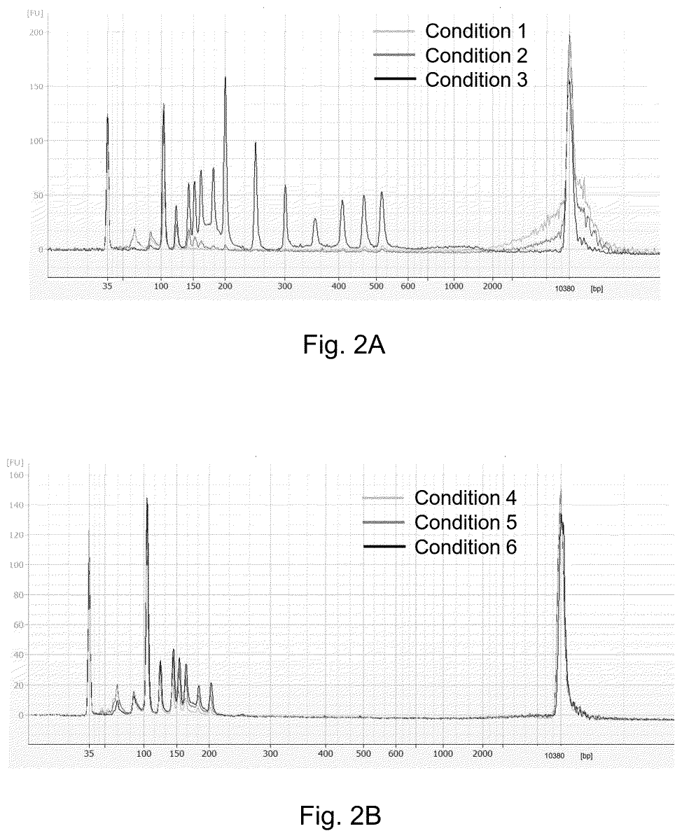

FIGS. 2A and 2B show electropherograms of DNA oligos recovery of plasma extracted from different volumes of top phase in the 2nd ATPS using direct fractionation (with Fractionation Buffer D1) and reverse fractionation (with Fractionation Buffer R1) respectively with smaller (˜150 bp) expected DNA cutoff value. The actual DNA cutoff values of purified DNA extracted in different top phase volumes of the 2^{nd} ATPS using direct fractionation (with Fractionation Buffer D1) or reverse fractionation (with Fractionation Buffer R1) are shown in Table 4.

TABLE 4
Actual DNA cutoff values of purified DNA extracted in
different top phase volumes of the 2^{nd} ATPS using direct
fractionation (with Fractionation Buffer D1) or reverse
fractionation with Fractionation Buffer R1)

| | Top | | | | |
|---|---|---|---|---|---|
| | phase | Fractionation | Fractionation | Actual DNA | |
| Condition | volume | formula | Method | cutoff value | |
| 1 | 100 uL | Fractionation | Direct | Low recovery | |
| | | Buffer D1 | Fractionation | of all DNA | |
| 2 | 150 uL | | | 121 | bp |
| 3 | 180 uL | | | >500 | bp |
| 4 | 100 uL | Fractionation | Reverse | 131 | bp |
| 5 | 150 uL | Buffer R1 | Fractionation | 147 | bp |
| 6 | 180 uL | | | 175 | bp |

Referring to FIG. 2A and Table 4, with direct fractionation, the recovery of larger DNA fragments (>100 bp) was significantly reduced when the top phase volume was small (condition 1, 100 μL), while significant increase in recovery of larger DNA oligos (100-200 bp) was observed when the top phase volume was large (condition 3, 180 μL). These results showed that the cutoff value is largely shifted when the top phase volume deviated from the standard (condition 2, 150 μL) using direct fractionation, demonstrating that the variation in sample volume greatly affects the stability and efficiency of DNA fractionation using direct fractionation.

Referring now to FIG. 2B and Table 4, with reverse fractionation, the difference between the sizes of DNA fragments recovered using reverse fractionation with various top phase volumes (100, 150 and 180 μL) was significantly lesser compared to direct fractionation, and the shift in DNA cutoff values was minimal, i.e. within an acceptable range of 131 bp-175 bp. As shown in FIG. 2B, the electropherogram patterns of conditions 4-6 using reverse fractionation with various top phase volumes are largely overlapped.

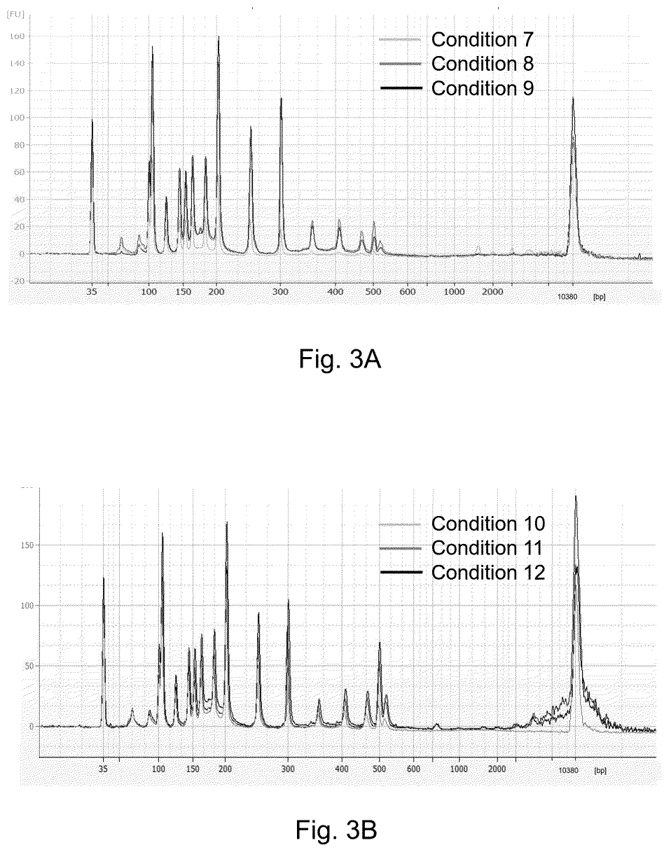

FIGS. 3A and 3B show electropherograms of DNA oligos recovery of plasma extracted from different volumes of top phase in the 2^{nd} ATPS using direct fractionation (with Fractionation Buffer D2) and reverse fractionation (with Fractionation Buffer R2) respectively with larger (˜300 bp) expected DNA cutoff value. The actual DNA cutoff values of purified DNA extracted in different top phase volumes of the 2^{nd} ATPS using direct fractionation (with Fractionation Buffer D2) or reverse fractionation with Fractionation Buffer R2) are shown in Table 5.

TABLE 5
Actual DNA cutoff values of purified DNA extracted in
different top phase volumes of the 2^{nd} ATPS using direct
fractionation (with Fractionation Buffer D2) or reverse
fractionation with Fractionation Buffer R2)

| | Top | | | | | |
|---|---|---|---|---|---|---|
| | phase | | Fractionation | Fractionation | Actual DNA | |
| Condition | volume | | formula | Method | cutoff value | |
| 7 | 100 | uL | Fractionation | Direct | 229 | bp |
| 8 | 150 | uL | Buffer D2 | Fractionation | 313 | bp |
| 9 | 180 | uL | | | 355 | bp |
| 10 | 100 | uL | Fractionation | Reverse | 281 | bp |
| 11 | 150 | uL | Buffer R2 | Fractionation | 297 | bp |
| 12 | 180 | uL | | | 356 | bp |

Referring to FIG. 3A and Table 5, with direct fractionation, the cutoff significantly shifted to smaller value (i.e. 229 bp) when the top phase volume was small (condition 7, 100 μL). Notably, when the top phase volume was large (condition 9, 180 μL), the recovery of small DNA fragments (<100 bp) was low (i.e. 31% relative to 150 uL condition), even that the recovery of larger DNA fragments was similar to that using top phase volume of 150 μL (condition 8).

Referring now to FIG. 3B and Table 5, with reverse fractionation, the difference between the sizes of DNA fragments recovered using reverse fractionation with various top phase volumes (100, 150 and 180 μL) was less significant compared to direct fractionation, and the shift in DNA cutoff values was minimal, i.e. within an acceptable range of 281 bp-356 bp. As shown in FIG. 3B, the electropherogram patterns of conditions 10-12 using reverse fractionation with various top phase volumes are largely overlapped.

Overall, the results demonstrate that reverse fractionation is unexpectedly more stable compared to direct fractionation towards different top phase volumes, in both DNA cutoff value and recovery of DNA, especially small DNA fragments. The high stability and efficiency of DNA fractionation using reverse fractionation method is advantageous for the isolation of small DNA fragments below a target size.

### Example 6

The following study was conducted to compare the stability and robustness of direct fractionation and reverse fractionation protocols for extraction of DNA form different sample types, i.e. plasma and urine. The stability and robustness of fractionation is important for a wide application. One of the variations is the sample type, as different components in the sample may affect the performance of fractionation to different degrees. To investigate the tolerance of direct and reverse fractionation towards different sample types, plasma and urine samples have been subjected to the study using the same direct and reverse fractionation buffers and protocols as described in Example 5, and their difference in performance was studied.

Materials and Methods

Plasma Cell Lysis

Each 2 mL of plasma was spiked with 100 fg of 145 bp dsDNA oligos, 80 ng 20 bp ladder (Jena Bioscience, Cat #M212) and 40 ng 50 bp ladder (Jena Bioscience, Cat #M-213). 160 μL of suitable lysis buffer and 60 μL of Proteinase K (28.5 mg/mL) was added into each 2 mL spiked plasma. The mixture was vortexed thoroughly then lysed for 15 minutes at a pre-heated 60° C. heat block.

Urine Pre-Treatment and Lysis

Urine samples were pre-treated with 200 μL of 0.1M EDTA per 10 mL urine sample, vortexed thoroughly and centrifuged at 3000×g for 10 minutes. This preserves the cell-free DNA (cfDNA) present in samples and prevents degradation over time. The supernatant was transferred to a new tube while the pellet was discarded. Each 2 mL of urine was spiked with 100 fg of 145 bp dsDNA oligos, 80 ng 20 bp ladder (Jena Bioscience, Cat #M212) and 40 ng 50 bp ladder (Jena Bioscience, Cat #M-213). Unwanted protein and cells present in pre-treated urine samples are lysed by adding 1200 μL of Proteinase K (28.57 mg/mL) and 4 mL of suitable lysis buffer solution to 40 mL of sample. The samples were then vortexed thoroughly till homogenous then left in a pre-heated 37° C. water bath to incubate for 15 minutes.

Two-Phase System

Lysed plasma and urine samples undergone two sequential aqueous two-phase systems (ATPSs) to isolate and concentrate DNA. 2.22 mL of lysed plasma or 2.26 mL of lysed urine was transferred to the first ATPS (polymer, salts and/or surfactant) and vortex mixed. The mixture was centrifuged at 2,300 rcf for 6 minutes. All salt-rich bottom phase (˜1 mL) where DNA was likely to partitioned to (also referred to as “first target-rich phase”), was transferred into a second ATPS (polymer, salts and/or surfactant) and thoroughly mixed. The mixture was then centrifuged at 7,000×g for 1 minute. Polymer-rich top phase (150 μL) which target DNA would have partitioned to (also referred to as “second target-rich phase”) was carefully extracted into an empty microcentrifuge tube for further purification. One sample set with plasma or urine sample would go through direct fractionation while another set would go through reverse fractionation protocol.

Direct Fractionation

Two direct fractionation buffer formulas which have different expected DNA cutoff sizes were used for each sample type: (i) Fractionation Buffer D1 for a smaller (around 150 bp) expected cutoff size, i.e. 3-7M guanidinium, 0.1-15% (w/v) of polymer, 0.01M-0.5M pH buffer and 0.01M-0.5M metal chelator; and (ii) Fractionation Buffer D2 for a larger (around 300 bp) expected cutoff size, i.e. 1-5M guanidinium, 0.1-15% (w/v) of polymer, 0.01M-0.5M pH buffer and 0.01M-0.5M metal chelator.

The steps to perform the direct fractionation for the separation and isolation of smaller DNA fragments from larger DNA fragments for the plasma and urine samples are the same or similar to those as discussed with respect to Example 5 above. For the sake of brevity and simplicity of the present disclosure, the discussion of the direct fractionation steps is not reproduced here.

Reverse Fractionation

Two reverse fractionation buffer formulas have been applied to each sample type targeting different expected DNA cutoff size: (i) Fractionation Buffer R1 for a smaller (around 150 bp) expected cutoff size, i.e. 1-5M guanidinium, 0.1-15% (w/v) of polymer, 0.01M-0.5M pH buffer and 0.01M-0.5M metal chelator; and (ii) Fractionation Buffer R2 for a larger (around 300 bp) expected cutoff size, i.e. 0.5-5.0M guanidinium, 0.01M-0.5M pH buffer and 0.01M-0.5M metal chelator.

The steps to perform the reverse fractionation for the separation and isolation of smaller DNA fragments from larger DNA fragments for the plasma and urine samples are the same or similar to those as discussed with respect to Example 5 above. For the sake of brevity and simplicity of the present disclosure, the discussion of the reverse fractionation steps is not reproduced here.

Purification of DNA

The steps to perform the purification of DNA for each sample are the same or similar to those as discussed with respect to Example 5 above. For the sake of brevity and simplicity of the present disclosure, the discussion of the purification steps is not reproduced here.

Detection of DNA

Recovery of different sizes of DNA oligos in extracted samples were quantified by electrophoresis. The gel mixture was prepared by Agilent™ High Sensitivity DNA Kit (Agilent, 5067-4626). 9 μL of gel-dye mix was distributed into each well on the microfluidic chip and 1 μL of purified DNA sample was added. Electrophoresis on a chip was performed by Agilent™ 2100 Bioanalyzer. The fluorescence signals of the reaction were collected and analyzed using the software Agilent™ Technologies 2100 Expert. The actual DNA cutoff value is estimated by the calculation method as described in Example 4b, i.e. calculating the value of base pair of the purified DNA sample in which the DNA recovery % is 70%.

The conditions of sample sets and compositions of the fractionation buffer formulas (for both direct and reverse fractionation) used in this study are summarized in Table 6 below.

TABLE 6
conditions of sample sets and compositions of the fractionation buffer formulas

| | Sample | Fractionation buffer | Fractionation | Expected |
|---|---|---|---|---|
| Condition | Matrix | formula | Method | Cutoff |
| 13 | 2 mL pre- | Fractionation Buffer D1: 3- | Direct | 150 bp |
| | treated | 7M guanidinium, 0.1-15% | Fractionation | |
| | urine | (w/v) of polymer, 0.01M- | | |
| | | 0.5M pH buffer and 0.01M- | | |
| | | 0.5M metal chelator | | |
| 14 | | Fractionation Buffer R1: 1- | Reverse | |
| | | 5M guanidinium , 0.1-15% | Fractionation | |
| | | (w/v) of polymer, 0.01M- | | |
| | | 0.5M pH buffer and 0.01M- | | |
| | | 0.5M metal chelator | | |
| 15 | | Fractionation Buffer D2: | Direct | 300 bp |
| | | 1-5M guanidinium, 0.1-15% | Fractionation | |
| | | (w/v) of polymer, 0.01M- | | |
| | | 0.5M pH buffer and 0.01M- | | |
| | | 0.5M metal chelator | | |
| 16 | | Fractionation Buffer R2: 0.5- | Reverse | |
| | | 5.0M guanidinium, 0.01M- | Fractionation | |
| | | 0.5M pH buffer and 0.01M- | | |
| | | 0.5M metal chelator | | |
| 17 | 2 mL | Fractionation Buffer D1: 3- | Direct | 150 bp |
| | K2EDTA | 7M guanidinium, 0.1-15% | Fractionation | |
| | plasma | (w/v) of polymer, 0.01M- | | |
| | | 0.5M pH buffer and 0.01M- | | |
| | | 0.5M metal chelator | | |
| 18 | | Fractionation Buffer R1: 1- | Reverse | |
| | | 5M guanidinium, 0.1-15% | Fractionation | |
| | | (w/v) of polymer, 0.01M- | | |
| | | 0.5M pH buffer and 0.01M- | | |
| | | 0.5M metal chelator | | |
| 19 | | Fractionation Buffer D2: | Direct | 300 bp |
| | | 1-5M guanidinium, 0.1-15% | Fractionation | |
| | | (w/v) of polymer, 0.01M- | | |
| | | 0.5M pH buffer and 0.01M- | | |
| | | 0.5M metal chelator | | |
| 20 | | Fractionation Buffer R2: 0.5- | Reverse | |
| | | 5.0M guanidinium, 0.01M- | Fractionation | |
| | | 0.5M pH buffer and 0.01M- | | |
| | | 0.5M metal chelator | | |

Results

Recovery of DNA

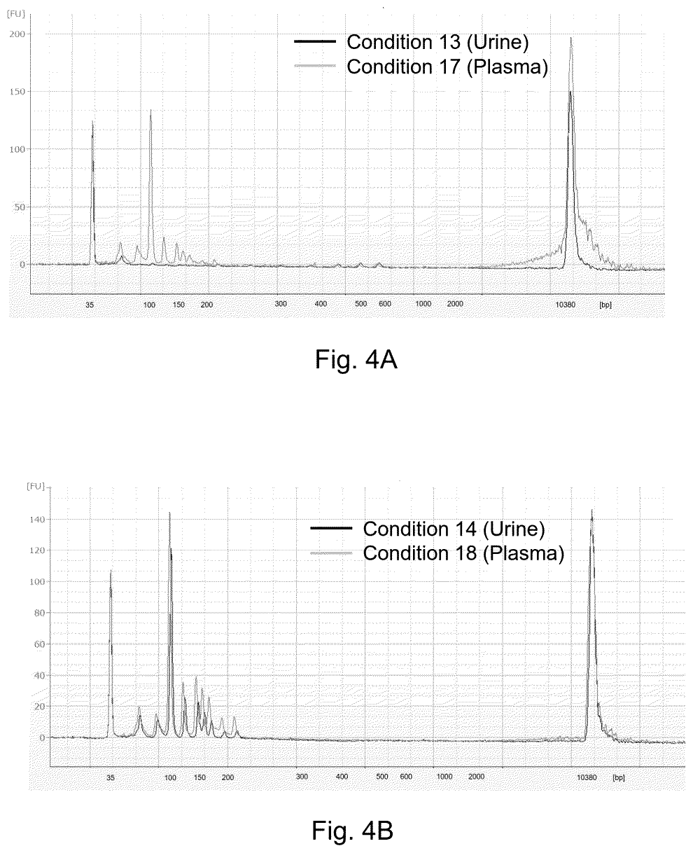

FIGS. 4A and 4B show electropherograms of DNA oligos recovery of different sample types, i.e. plasma and urine, using direct fractionation (with Fractionation Buffer D1) and reverse fractionation (with Fractionation Buffer R1) respectively with smaller (˜150 bp) expected DNA cutoff value. The actual DNA cutoff value of purified DNA extracted in different sample types using direct fractionation (with Fractionation Buffer D1) or reverse fractionation (with Fractionation Buffer R1) is shown in Table 7.

TABLE 7
Actual DNA cutoff values of purified DNA extracted in different
sample types using direct fractionation (with Fractionation Buffer
D1) or reverse fractionation with Fractionation Buffer R1)

| | Sample | Fractionation | Fractionation | Actual DNA |
|---|---|---|---|---|
| Condition | Matrix | buffer formula | Method | cutoff value |
| 13 | 2 mL pre- | Fractionation | Direct | Low DNA |
| | treated | Buffer D1 | Fractionation | recovery |
| 14 | urine | Fractionation | Reverse | 125 bp |
| | | Buffer R1 | Fractionation | |
| 17 | 2 mL | Fractionation | Direct | 121 bp |
| | K2EDTA | Buffer D1 | Fractionation | |
| 18 | plasma | Fractionation | Reverse | 147 bp |
| | | Buffer R1 | Fractionation | |

Referring to FIG. 4A and Table 7, with a smaller (˜150 bp) expected cutoff value, direct fractionation performed well with plasma sample, but only a little DNA of small size (<50 bp) was found to be recovered in urine sample, demonstrating the incapability of direct fractionation in accommodating various sample type input.

Referring now to FIG. 4B and Table 7, with reverse fractionation, the difference between the sizes of DNA fragments recovered using reverse fractionation with plasma and urine was less significant compared to direct fractionation, and the difference in DNA cutoff values (147 bp for plasma, 125 bp for urine) was small, demonstrating the stability of the reverse fractionation and wider usability of reverse fractionation for various samples types.

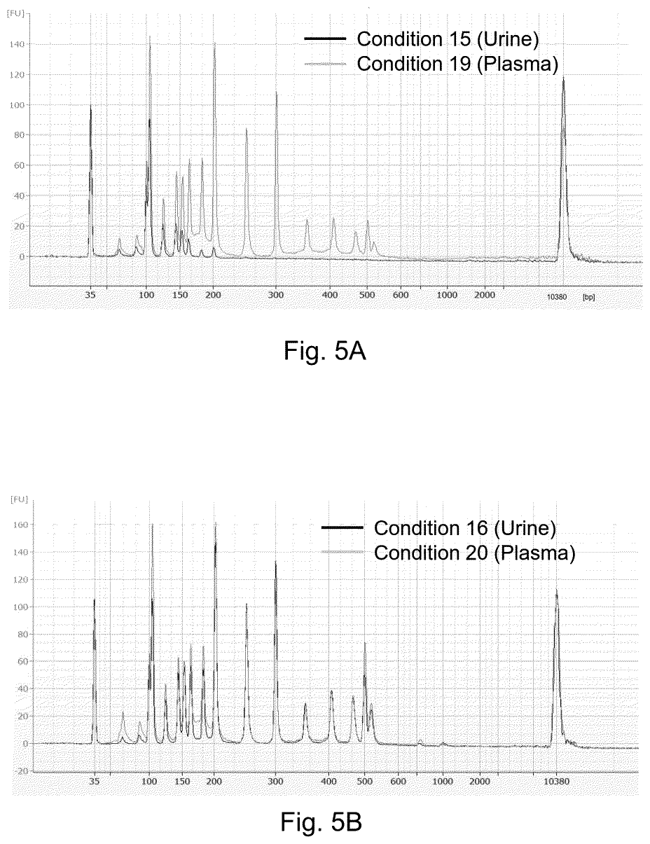

FIGS. 5A and 5B show electropherograms of DNA oligos recovery of plasma extracted from different sample types using direct fractionation (with Fractionation Buffer D2) and reverse fractionation (with Fractionation Buffer R2) respectively with larger (˜300 bp) expected DNA cutoff value. The actual DNA cutoff values of purified DNA extracted in different sample types using direct fractionation (with Fractionation Buffer D2) or reverse fractionation (with Fractionation Buffer R2) are shown in Table 8.

TABLE 8
Actual DNA cutoff values of purified DNA extracted in different
sample types using direct fractionation (with Fractionation Buffer
D2) or reverse fractionation with Fractionation Buffer R2)

| | Sample | Fractionation | Fractionation | Actual DNA |
|---|---|---|---|---|
| Condition | Matrix | buffer formula | Method | cutoff value |
| 15 | 2 mL pre- | Fractionation | Direct | 138 bp |
| | treated | Buffer D2 | Fractionation | |
| 16 | urine | Fractionation | Reverse | 469 bp |
| | | Buffer R2 | Fractionation | |
| 19 | 2 mL | Fractionation | Direct | 313 bp |
| | K2EDTA | Buffer D2 | Fractionation | |
| 20 | plasma | Fractionation | Reverse | 332 bp |
| | | Buffer R2 | Fractionation | |

Referring to FIG. 5A and Table 8, with direct fractionation, the cutoff significantly shifted to a much smaller value (i.e. 138 bp) when urine was used instead of plasma, demonstrating that direct fractionation could result in an excessive removal of target DNA when changing sample type which may be detrimental to downstream assay.

Referring to FIG. 5B, with reverse fractionation, the electropherogram patterns of DNA fragments recovered using reverse fractionation with different sample types, i.e. plasma and urine, are largely overlapping, indicating that the size fractionation and target DNA extracted are generally consistent over different sample types. Although the DNA cutoff values in the urine sample shifted to a slightly larger value, this will not result in an excessive removal of target DNA (as compared to direct fractionation), and therefore will be more suitable for downstream assays.

Overall, these results demonstrated that reverse fractionation is more stable towards different sample types, which allowed a broader application compared to direct fractionation.

### Example 7

The following study was conducted to assess the estimated DNA cutoff sizes using reverse fractionation with different fractionation buffer formulas.

Materials and Methods

Plasma Cell Lysis

Each 2 mL of plasma was spiked with 100 fg of 145 bp dsDNA oligos and 80 ng 20 bp ladder (Jena Bioscience, Cat #M212). 160 μL of suitable lysis buffer and 60 μL of Proteinase K (28.5 mg/mL) was added into each 2 mL spiked plasma. The mixture was vortexed thoroughly then lysed for 15 minutes at a pre-heated 60° C. heat block.

Two-Phase System

Lysed plasma samples undergone two sequential aqueous two-phase systems (ATPSs) to isolate and concentrate DNA. The steps to perform the sequential ATPSs for the plasma samples are the same or similar to those as discussed with respect to Example 5 above. For the sake of brevity and simplicity of the present disclosure, the discussion of the sequential ATPSs steps is not reproduced here. The samples collected after ATPS steps would go through reverse fractionation protocol with different reverse fractionation buffer formulas.

Reverse Fractionation

Different reverse fractionation buffer formulas were used and summarized in Table 9 below. The steps to perform the reverse fractionation for the separation and isolation of smaller DNA fragments from larger DNA fragments for the plasma samples are the same or similar to those as discussed with respect to Example 5 above. For the sake of brevity and simplicity of the present disclosure, the discussion of the reverse fractionation steps is not reproduced here.

TABLE 9
Reverse fractionation buffer formulas

| Reverse | | | | |
|---|---|---|---|---|
| Fractionation | chaotropic | | | metal |
| buffer formula | agent | polymer | pH buffer | chelator |
| Buffer R-015 | 2.3-5.4M | 0.1-15% | 0.01M- | 0.01M- |
| | Guanidinium | (w/v) | 0.5M pH | 0.5M metal |
| | | polymer | buffer | chelator |
| Buffer R-016 | 2.2-5.0M | 0.1-15% | 0.01M- | 0.01M- |
| | Guanidinium | (w/v) | 0.5M pH | 0.5M metal |
| | | polymer | buffer | chelator |
| Buffer R-017 | 2.0-4.6M | 0.1-15% | 0.01M- | 0.01M- |
| | Guanidinium | (w/v) | 0.5M pH | 0.5M metal |
| | | polymer | buffer | chelator |
| Buffer R-018 | 1.8-4.2M | 0.1-15% | 0.01M- | 0.01M- |
| | Guanidinium | (w/v) | 0.5M pH | 0.5M metal |
| | | polymer | buffer | chelator |
| Buffer R-019 | 1.6-3.8M | 0.1-15% | 0.01M- | 0.01M- |
| | Guanidinium | (w/v) | 0.5M pH | 0.5M metal |
| | | polymer | buffer | chelator |
| Buffer R-020 | 1.4-3.4M | 0.1-15% | 0.01M- | 0.01M- |
| | Guanidinium | (w/v) | 0.5M pH | 0.5M metal |
| | | polymer | buffer | chelator |
| Buffer R-021 | 1.3-2.9M | 0.1-15% | 0.01M- | 0.01M- |
| | Guanidinium | (w/v) | 0.5M pH | 0.5M metal |
| | | polymer | buffer | chelator |
| Buffer R-022 | 1.2-2.8M | 0.1-15% | 0.01M- | 0.01M- |
| | Guanidinium | (w/v) | 0.5M pH | 0.5M metal |
| | | polymer | buffer | chelator |

Purification of DNA

The steps to perform the purification of DNA for each sample are the same or similar to those as discussed with respect to Example 5 above. For the sake of brevity and simplicity of the present disclosure, the discussion of the purification steps is not reproduced here.

Detection of DNA

Recovery of different sizes of DNA oligos in extracted samples were quantified by electrophoresis. The steps to perform the detection of DNA for each sample are the same or similar to those as discussed with respect to Example 5 above. For the sake of brevity and simplicity of the present disclosure, the discussion of the detection steps is not reproduced here. The recovery % and the estimated DNA cutoff value (also referred to as “actual DNA cutoff value” in some embodiments) is calculated by the method as described in Example 4b, i.e. calculating the value of base pair of the purified DNA sample in which the DNA recovery % is 70%.

Results

Recovery of DNA

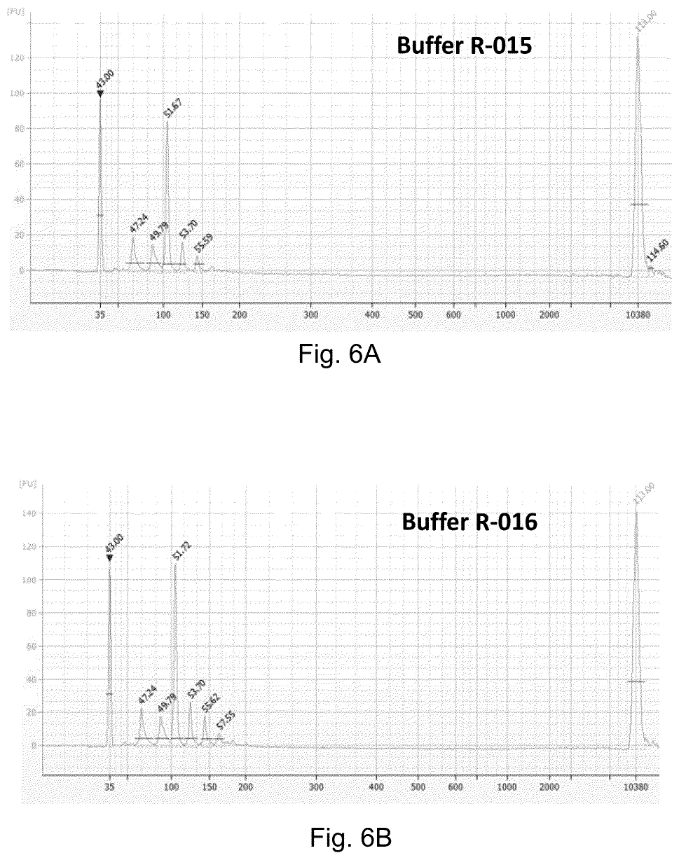

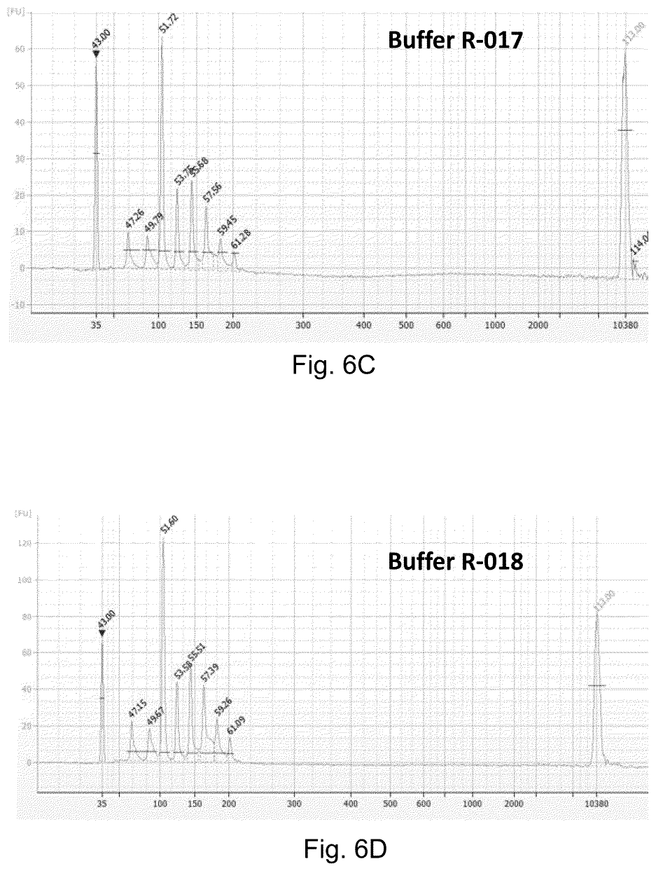

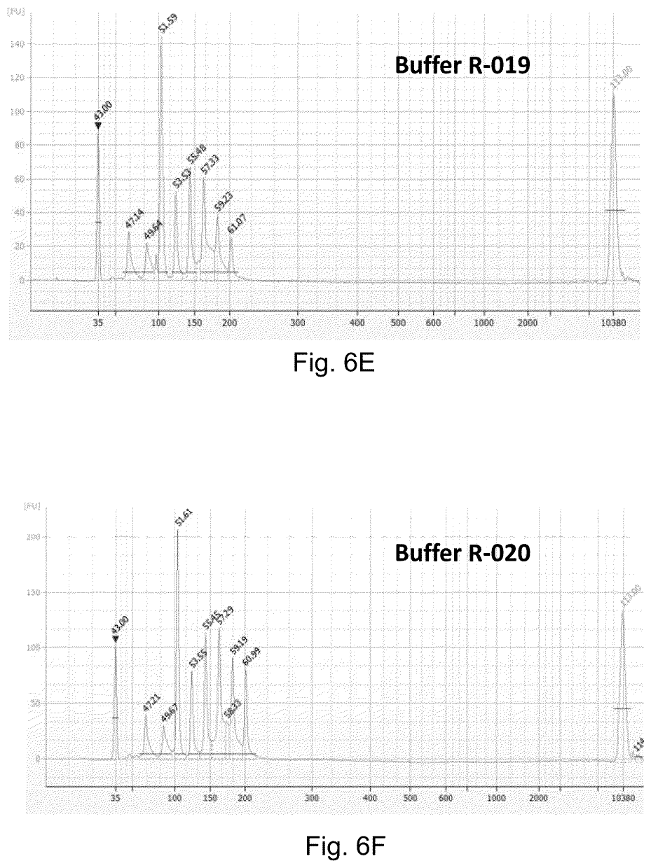

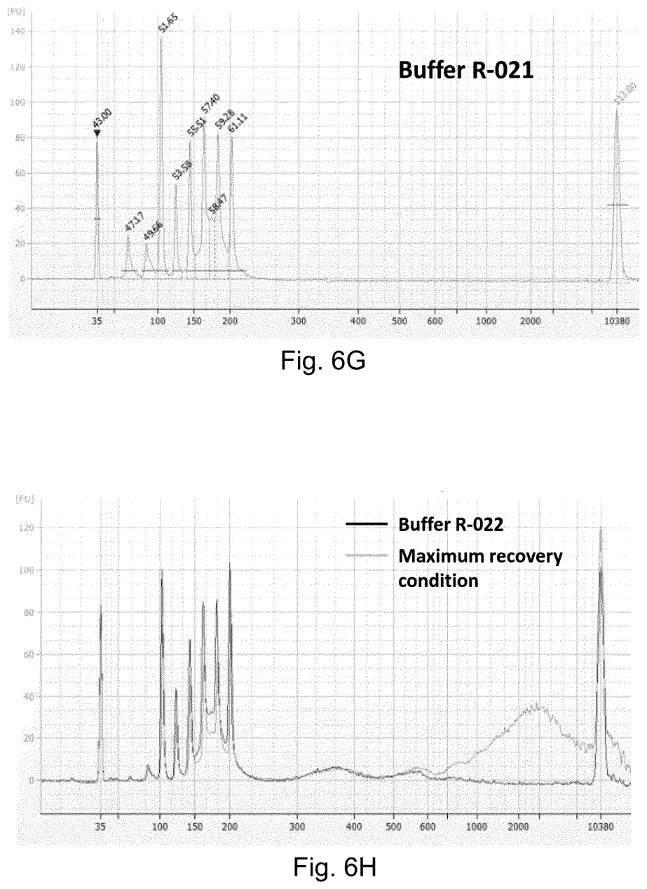

FIGS. 6A-6H show electropherograms of DNA oligos recovery of plasma sample using reverse fractionation with different reverse fractionation buffer formula R-015 to R-021 respectively. The estimated DNA cutoff value and recovery % of different sizes of DNA extracted using different reverse fractionation formula is shown in Table 10.

TABLE 10
Estimated DNA cutoff value and recovery % of different sizes of DNA
extracted using different reverse fractionation buffer formulas

| | Buffer R-015 | | Buffer R-016 | | Buffer R-017 | |
|---|---|---|---|---|---|---|
| | | Estimated | | Estimated | | Estimated |
| Base | Recovery | cutoff (70% | Recovery | cutoff (70% | Recovery | cutoff (70% |
| pair | % | recovery) | % | recovery) | % | recovery) |
| 100 | 100% | 108 bp | 100% | 116 bp | 108% | 134 bp |
| 120 | 48% | | 64% | | 85% | |
| 140 | 20% | | 31% | | 63% | |
| 160 | 0% | | 10% | | 37% | |
| 180 | 0% | | 0% | | 17% | |
| 200 | 0% | | 0% | | 7% | |
| | Buffer R-018 | | Buffer R-019 | | Buffer R-020 | |
| | | Estimated | | Estimated | | Estimated |
| Base | Recovery | cutoff (70% | Recovery | cutoff (70% | Recovery | cutoff (70% |
| pair | % | recovery) | % | recovery) | % | recovery) |
| 100 | 100% | 149 bp | 100% | 156 bp | 111% | 171 bp |
| 120 | 90% | | 92% | | 96% | |
| 140 | 86% | | 91% | | 93% | |
| 160 | 52% | | 65% | | 82% | |
| 180 | 25% | | 37% | | 61% | |
| 200 | 11% | | 18% | | 42% | |

| | Buffer R-021 | | | |
|---|---|---|---|---|
| | | Estimated | Buffer R-022 | |
| Base | Recovery | cutoff (70% | Recovery | |
| pair | % | recovery) | % | Estimated cutoff (70% recovery) |
| 100 | 100% | 192 bp | N/A | around 500 bp |
| 120 | 100% | | N/A | (the 500 bp cutoff is inferred through |
| 140 | 100% | | N/A | comparing the cfDNA bumps when |
| 160 | 91% | | N/A | compared to a maximum recovery |
| 180 | 79% | | N/A | condition, i.e. the recovery of DNA |
| 200 | 63% | | N/A | ladder original profile without any |
| | | | | size fractionation step) |

Referring to 6A-6H and Table 10, the results showed that the reverse fractionation method can be performed by using a wide range of reverse fractionation buffer formula. The results also demonstrated that by altering the concentration of chaotropic agent, pH buffer and/or metal chelator in the fractionation buffer, the DNA cutoff value can be controlled (for example, from around 100 bp to around 500 bp), which allows for a precise size selection of the recovered DNA molecules.

### NUMBERED EMBODIMENTS

### Set 1:

Embodiment 1. A method for concentrating and purifying one or more target analytes from a sample solution, comprising the steps of: (a) adding a sample solution containing the target analyte(s) to a first aqueous two-phase system (ATPS) to form a mixture that partitions into a first phase and a second phase, wherein the target analyte(s) are concentrated in the first phase; (b) isolating the first phase containing the concentrated target analyte(s), thereby resulting in a concentrated solution; (c) applying magnetic beads to the concentrated solution, such that the magnetic beads bind the target analyte(s) to form a beads-analyte complex; and (d) recovering the target analyte(s) from the beads-analyte complex, resulting in a final solution containing the target analyte(s) that is concentrated and purified.

Embodiment 2. The method of embodiment 1, wherein the step (b) further comprises the following steps: (i) adding the isolated first phase that is concentrated with the target analyte(s) to a second ATPS to form a second mixture that partitions into a third phase and a fourth phase, wherein the target analyte(s) are concentrated in the third phase; and (ii) isolating the third phase containing the concentrated target analyte(s) to form the concentrated solution in step (b) for step (c).

Embodiment 3. The method of any of the preceding embodiments, wherein the concentrated solution of step (b) is mixed with a binding buffer, wherein the binding buffer comprises at least one chaotropic agent selected from n-butanol, ethanol, guanidinium chloride, guanidinium thiocyanate, lithium perchlorate, lithium acetate, magnesium chloride, phenol, 2-propanol, sodium dodecyl sulfate, thiourea, and urea, thereby resulting in the concentration solution for step (c).

Embodiment 4. The method of any one of the preceding embodiments, wherein the step (d) further comprises the steps of: (i) mixing the beads-analyte complex with a fractionation buffer comprising a polymer, a salt, a surfactant, a chaotropic agent or combinations thereof to form a fractionation solution, such that the target analyte(s) below a target size are released from the beads-analyte complex into the fractionation solution; (ii) immobilizing the beads-analyte complex using a magnetic stand; and (iii) isolating the target analyte(s) below the target size in the fractionation solution from the immobilized beads-analyte complex.

Embodiment 5. The method of embodiment 4, wherein the step (d) further comprises the steps of: (iv) adding the isolated target analyte(s) below the target size to a second binding buffer, wherein the second binding buffer comprises at least one chaotropic agent selected from n-butanol, ethanol, guanidinium chloride, guanidinium thiocyanate, lithium perchlorate, lithium acetate, magnesium chloride, phenol, 2-propanol, sodium dodecyl sulfate, thiourea, and urea; (v) applying magnetic beads to a mixture of the isolated target analyte(s) below the target size and the second binding buffer, wherein the magnetic beads bind the target analyte(s) below the target size to form a second beads-analyte complex; and (vi) recovering the target analyte(s) from the second beads-analyte complex.

Embodiment 6. The method of any one of the preceding embodiments, further comprising the step of: (e) subjecting said final solution to a diagnostic assay for detection and quantification of said target analyte(s).

Embodiment 7. The method of any one of the preceding embodiments, wherein the target analyte(s) is selected from the group consisting of nucleic acids, a protein, an antigen, a biomolecule, a sugar moiety, a lipid, a sterol, and combinations thereof.

Embodiment 8. The method of any one of the preceding embodiments, wherein the target analyte(s) is DNA.

Embodiment 9. The method of any preceding embodiments, wherein the target analyte(s) is cell-free DNA or circulating tumor DNA.

Embodiment 10. The method of any of the preceding embodiments, wherein the first ATPS comprises first ATPS components capable of forming the first phase and the second phase when the first ATPS components are dissolved in an aqueous solution, wherein the first ATPS components are selected from the group consisting of polymers, salts, surfactants, and combinations thereof.

Embodiment 11. The method of any one of embodiments 2-10, wherein the second ATPS comprises second ATPS components capable of forming the third phase and the fourth phase when the second ATPS components are dissolved in an aqueous solution, wherein the second ATPS components are selected from the group consisting of polymers, salts, surfactants, and combinations thereof.

Embodiment 12. The method of embodiment 10 or 11, wherein said polymers dissolve in the aqueous solution at a concentration of 4%-84% (w/w).

Embodiment 13. The method of any one of embodiments 10-12, wherein said polymers are selected from the group consisting of polyalkylene glycols (PEGs), such as hydrophobically modified polyalkylene glycols, poly(oxyalkylene)polymers, poly(oxyalkylene)copolymers, such as hydrophobically modified poly(oxyalkylene)copolymers, polyvinyl pyrrolidone, polyvinyl alcohol, polyvinyl caprolactam, polyvinyl methylether, alkoxylated surfactants, alkoxylated starches, alkoxylated cellulose, alkyl hydroxyalkyl cellulose, silicone-modified polyethers, and poly N-isopropylacrylamide and copolymers thereof. The method of any one of the preceding embodiments, wherein the polymer is selected from the group consisting of polyether, polyimine, polyalkylene glycol, vinyl polymer, alkoxylated surfactant, polysaccharides, alkoxylated starch, alkoxylated cellulose, alkyl hydroxyalkyl cellulose, polyether-modified silicones, polyacrylamide, polyacrylic acid and copolymer thereof. The method of any one of the preceding embodiments, wherein the polymer is selected from the group consisting of dipropylene glycol, tripropylene glycol, polyethylene glycol, polypropylene glycol, poly(ethylene glycol-propylene glycol), poly(ethylene glycol-ran-propylene glycol), polyvinyl pyrrolidone, polyvinyl alcohol, polyvinyl caprolactam, polyvinyl methylether, dextran, carboxymethyl dextran, dextran sulfate, hydroxypropyl dextran, starch, carboxymethyl cellulose, polyacrylic acid, hydroxypropyl cellulose, methyl cellulose, ethylhydroxyethylcellulose, maltodextrin, polyethyleneimine, poly N-isopropylacrylamide and copolymers thereof. The method of any one of the preceding embodiments, wherein the polymer is selected from the group consisting of dipropylene glycol, tripropylene glycol, polyethylene glycol, polypropylene glycol, poly(ethylene glycol-propylene glycol), poly(ethylene glycol-ran-propylene glycol), polyvinyl pyrrolidone, polyvinyl alcohol, polyvinyl caprolactam, polyvinyl methylether and poly N-isopropylacrylamide. The method of any one of the preceding embodiments, wherein the polymer is selected from the group consisting of polyacrylamide, polyacrylic acid and copolymers thereof. The method of any one of the preceding embodiments, wherein the polymer is selected from the group consisting of dextran, carboxymethyl dextran, dextran sulfate, hydroxypropyl dextran and starch. The method of any one of the preceding embodiments, wherein the polymer has an average molecular weight in the range of 200-1,000 Da, 200-35,000 Da, 425-2,000 Da, 400-35,000 Da, 980-12,000 Da, or 3,400-5,000,000 Da. The method of any one of the preceding embodiments, wherein the polymer comprises ethylene oxide and propylene oxide units, and the polymer has an EO:PO ratio of 90:10 to 10:90.

Embodiment 14.

Embodiment 15. The method of any one of embodiments 10-14, wherein said salts dissolve in the aqueous solution at a concentration of 1%-80% (w/w).

Embodiment 16. The method of any one of embodiments 10-15, wherein said salts dissolve in the aqueous solution at a concentration of 8%-80% (w/w).

Embodiment 17. The method of any one of embodiments 10-16, wherein said salts are selected from the group consisting of kosmotropic salts, chaotropic salts, inorganic salts containing cations such as straight or branched trimethyl ammonium, triethyl ammonium, tripropyl ammonium, tributyl ammonium, tetramethyl ammonium, tetraethyl ammonium, tetrapropyl ammonium and tetrabutyl ammonium, and anions such as phosphates, sulphate, nitrate, chloride and hydrogen carbonate, NaCl, Na₃PO₄, K₃PO₄, Na₂SO₄, potassium citrate, (NH₄)₂SO₄, sodium citrate, sodium acetate, ammonium acetate, a magnesium salt, a lithium salt, a sodium salt, a potassium salt, a cesium salt, a zinc salt, an aluminum salt, a bromide salt, an iodide salt, a fluoride salt, a carbonate salt, a sulfate salt, a citrate salt, a carboxylate salt, a borate salt, a phosphate salt, potassium phosphate and ammonium sulfate.

Embodiment 18. The method of any one of embodiments 10-17, wherein said surfactants dissolve in the aqueous solution at a concentration of 0.05%-10% (w/w).

Embodiment 19. The method of any one of embodiments 10-18, wherein said surfactants dissolve in the aqueous solution at a concentration of 0.05%-9.8% (w/w).

Embodiment 20. The method of any one of embodiments 10-19, wherein said surfactants are selected from the group consisting of Triton-X, Triton-114, Igepal CA-630 and Nonidet P-40, anionic surfactants, such as carboxylates, sulphonates, petroleum sulphonates, alkylbenzenesulphonates, naphthalenesulphonates, olefin sulphonates, alkyl sulphates, sulphates, sulphated natural oils & fats, sulphated esters, sulphated alkanolamides, alkylphenols, ethoxylated and sulphated, nonionic surfactants, such as ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, polyoxyethylene fatty acid amides, cationic surfactants, such as quaternary ammonium salts, amines with amide linkages, polyoxyethylene alkyl & alicyclic amines, n,n,n′,n′ tetrakis substituted ethylenediamines, 2-alkyl 1-hydroxethyl 2-imidazolines, and amphoteric surfactants, such as n-coco 3-aminopropionic acid/sodium salt, n-tallow 3-iminodipropionate, disodium salt, n-carboxymethyl n dimethyl n-9 octadecenyl ammonium hydroxide, n-cocoamidethyl n hydroxyethylglycine, and sodium salt.

Embodiment 21. The method of any of the preceding embodiments, wherein the step (a) further comprises the steps of: (i) embedding a porous material with components capable of forming the first ATPS; and (ii) contacting the sample solution with the porous material embedded with the components, wherein said components form the first phase and the second phase when the sample solution travels through said porous material.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

1. A method for isolating target nucleic acids below a target size from a sample comprising nucleic acid components; comprising the steps of:
(a) preparing a sample solution from the sample;
(b) contacting a plurality of beads with the sample solution, wherein the nucleic acid components bind to the plurality of beads to form a beads-analyte complex;
(c) mixing the beads-analyte complex with a fractionation buffer comprising at least one chaotropic agent to form a bulk fractionation solution, wherein the target nucleic acids below the target size are released from the beads-analyte complex into the bulk fractionation solution;
(d) immobilizing the beads-analyte complex; and
(e) separating the bulk fractionation solution comprising the isolated target nucleic acids below the target size from the immobilized beads-analyte complex.

2. The method of claim 1, wherein the step (a) further comprises
(a1) adding the sample to a first aqueous two-phase system (ATPS) to form a mixture that partitions into a first target-rich phase and a first target-poor phase, wherein the nucleic acid components are concentrated in the first target-rich phase; and
(a2) isolating the first target-rich phase containing the concentrated nucleic acid components, resulting in the sample solution.

3. The method of claim 2, wherein the step (a) further comprises the following steps after step (a2):
(a3) adding the sample solution in step (a2) to a second ATPS to form a second mixture that partitions into a second target-rich phase and a second target-poor phase, wherein the nucleic acid components are concentrated in the second target-rich phase; and
(a4) isolating the second target-rich phase containing the concentrated nucleic acid components to form the sample solution in step (a).

4. The method of claim 1, wherein the plurality of beads and the sample solution of step (a) are mixed with a binding buffer prior to the step (b), wherein the binding buffer comprises at least one chaotropic agent.

5. The method of claim 1, wherein
the step (e) further comprises the steps of:
(e1) mixing the bulk fractionation solution with a target binding buffer and a plurality of second beads, such that the plurality of second beads bind the target nucleic acids below the target size to form a second beads-analyte complex, wherein the target binding buffer comprises at least one chaotropic agent; and
(e2) recovering the target nucleic acids below the target size from the second beads-analyte complex.

6. The method of claim 1, wherein the plurality of beads is magnetic beads, silica-based beads, carboxyl beads, hydroxyl beads, amine-coated beads, or any combination thereof.

7. The method of claim 5, wherein the plurality of second beads is magnetic beads, silica-based beads, carboxyl beads, hydroxyl beads, amine-coated beads, or any combination thereof.

8. The method of claim 1, wherein
the plurality of beads is magnetic beads, and the step (b) further comprises the steps of:
(b1) immobilizing the beads-analyte complex by applying a magnetic field to separate the beads-analyte complex from a bulk supernatant;
(b2) removing the bulk supernatant; and
(b3) removing the magnetic field and proceeding to step (c).

9. The method of claim 5, wherein the plurality of second beads is magnetic beads, and the target nucleic acids recovery of step (e2) further comprises steps of:
(i) immobilizing the second beads-analyte complex by applying a first magnetic field to separate the second beads-analyte complex from a first supernatant;
(ii) removing the first supernatant;
(iii) washing the immobilized second beads-analyte complex with a washing buffer;
(iv) discarding the washing buffer;
(v) removing the first magnetic field;
(vi) mixing the second beads-analyte complex with an elution buffer to form a bulk elution solution, wherein the target nucleic acids below the target size are separated from the magnetic beads in the second beads-analyte complex and released into the bulk elution solution;
(vii) immobilizing the magnetic beads by applying a second magnetic field;
(viii) collecting the bulk elution solution comprising the isolated target nucleic acids below the target size.

10. The method of claim 1, further comprising the step of:
(f) subjecting the isolated target nucleic acids to a diagnostic assay for detection, quantification, characterization, or combinations thereof, of the target nucleic acids.

11. The method of claim 1, wherein the at least one chaotropic agent of the fractionation buffer is selected from the group consisting of thiocyanate, isothiocyanate, perchlorate, acetate, trichloroacetate, trifluoroacetate, chloride, and iodide.

12. The method of claim 1, wherein the at least one chaotropic agent of the fractionation buffer is selected from the group consisting of guanidinium hydrochloride (GHCl), guanidinium thiocyanate, guanidinium isothiocyanate (GITC), sodium thiocyanate, sodium iodide, sodium perchlorate, sodium trichloroacetate, sodium trifluroacetate, lithium perchlorate, lithium acetate, magnesium chloride, phenol, 2-propanol, thiourea, and urea.

13. The method of claim 1, wherein the at least one chaotropic agent has a concentration of around 1.5-8M in the fractionation buffer.

14. The method of claim 13, wherein the at least one chaotropic agent is present at a concentration of around 1.8-3.9M in the fractionation buffer.

15. The method of claim 14, wherein the at least one chaotropic agent is present at a concentration of around 1.8-3.0M in the fractionation buffer.

16. The method of claim 1, wherein the fractionation buffer further comprises at least one polymer selected from the group consisting of polyvinyl alcohol, polyethylene glycol, polypropylene glycol, dextran, poly(ethylene glycol-ran-propylene glycol), pluronics, polyvinylpyrolidone, and polyacrylate.

17. The method of claim 16, wherein the at least one polymer is present at a concentration of around 0.1-15% (w/w) in the fractionation buffer.

18. The method of claim 16, wherein the at least one polymer is present at a concentration of around 1.0-5.0% (w/w) in the fractionation buffer.

19. The method of claim 16, wherein the at least one polymer has an average molecular weight range from 100 to 35,000 Da.

20. The method of claim 1, wherein the fractionation buffer further comprises one or more of a pH buffer, metal chelator, or combination thereof.

21. The method of claim 1, wherein the nucleic acid components and/or the target nucleic acids are DNA, RNA or combinations thereof.

22. The method of claim 21, wherein the nucleic acid components and/or the target nucleic acids are cDNA, plasmid DNA, cell-free DNA (cfDNA), circulating tumor DNA (ctDNA), circulating fetal DNA, micro RNA (miRNA), messenger RNA (mRNA), transfer RNA (tRNA), ribosomal RNA (rRNA), or combinations thereof.

23. The method of claim 3, wherein the first ATPS comprises first ATPS components capable of forming the first target-rich phase and the first target-poor phase when the first ATPS components are dissolved in an aqueous solution, wherein the first ATPS components are selected from the group consisting of a polymer, salt, surfactant, and combinations thereof.

24. The method of claim 23, wherein the second ATPS comprises second ATPS components capable of forming the second target-rich phase and the second target-poor phase when the second ATPS components are dissolved in an aqueous solution, wherein the second ATPS components are selected from the group consisting of a polymer, salt, surfactant, and combinations thereof.

25. The method of claim 24, wherein said polymer dissolves in the aqueous solution at a concentration of 0.5%-80% (w/v).

26. The method of claim 24, wherein the polymer is selected from the group consisting of polyether, polyimine, polyalkylene glycol, vinyl polymer, alkoxylated surfactant, polysaccharides, alkoxylated starch, alkoxylated cellulose, alkyl hydroxyalkyl cellulose, polyether-modified silicones, polyacrylamide, polyacrylic acid and a copolymer thereof.

27. The method of claim 24, wherein the polymer is selected from the group consisting of dipropylene glycol, tripropylene glycol, polyethylene glycol, polypropylene glycol, poly(ethylene glycol-propylene glycol), poly(ethylene glycol-ran-propylene glycol), polyvinyl pyrrolidone, polyvinyl alcohol, polyvinyl caprolactam, polyvinyl methylether, dextran, carboxymethyl dextran, dextran sulfate, hydroxypropyl dextran, starch, carboxymethyl cellulose, polyacrylic acid, hydroxypropyl cellulose, methyl cellulose, ethylhydroxyethylcellulose, maltodextrin, polyethyleneimine, poly N-isopropylacrylamide and copolymers thereof.

28. The method of claim 24, wherein the polymer is selected from the group consisting of dipropylene glycol, tripropylene glycol, polyethylene glycol, polypropylene glycol, poly(ethylene glycol-propylene glycol), poly(ethylene glycol-ran-propylene glycol), polyvinyl pyrrolidone, polyvinyl alcohol, polyvinyl caprolactam, polyvinyl methylether and poly N-isopropylacrylamide.

29. The method of claim 24, wherein the polymer is selected from the group consisting of polyacrylamide, polyacrylic acid and copolymers thereof.

30. The method of claim 24, wherein the polymer is selected from the group consisting of dextran, carboxymethyl dextran, dextran sulfate, hydroxypropyl dextran and starch.

31. The method of claim 24, wherein the polymer has an average molecular weight in the range of 200-1,000 Da, 200-35,000 Da, 425-2,000 Da, 400-35,000 Da, 980-12,000 Da, or 3,400-5,000,000 Da.

32. The method of claim 24, wherein the polymer comprises ethylene oxide and propylene oxide units, and the polymer has an EO:PO ratio of 90:10 to 10:90.

33. The method of claim 24, wherein said salt is dissolved in the aqueous solution at a concentration of 0.1%-80% (w/w).

34. The method of claim 33, wherein said salt is dissolved in the aqueous solution at a concentration of 0.1%-50% (w/w).

35. The method of claim 33, wherein the salt comprises a cation selected from the group consisting of sodium, potassium, calcium, ammonium, lithium, magnesium, aluminium, cesium, barium, straight or branched trimethyl ammonium, triethyl ammonium, tripropyl ammonium, tributyl ammonium, tetramethyl ammonium, tetraethyl ammonium, tetrapropyl ammonium and tetrabutyl ammonium.

36. The method of claim 33, wherein the salt comprises an anion selected from the group consisting of phosphate, hydrogen phosphate, dihydrogen phosphate, sulfate, sulfide, sulfite, hydrogen sulfate, carbonate, hydrogen carbonate, acetate, nitrate, nitrite, sulfite, chloride, fluoride, chlorate, perchlorate, chlorite, hypochlorite, bromide, bromate, hypobromite, iodide, iodate, cyanate, thiocyanate, isothiocyanate, oxalate, formate, chromate, dichromate, permanganate, hydroxide, hydride, citrate, borate, and tris.

37. The method of claim 33, wherein the salt is selected from the group consisting of aluminum chloride, aluminum phosphate, aluminum carbonate, magnesium chloride, magnesium phosphate, and magnesium carbonate.

38. The method of claim 33, wherein the salt is selected from the group consisting of NaCl, KCl, NH₄Cl, Na₃PO₄, K₃PO₄, Na₂SO₄, K₂HPO₄, KH₂PO₄, Na₂HPO₄, NaH₂PO₄, (NH₄)₃PO₄, (NH₄)₂HPO₄, NH₄H₂PO₄, potassium citrate, (NH₄)₂SO₄, sodium citrate, sodium acetate, magnesium acetate, sodium oxalate, sodium borate, and ammonium acetate.

39. The method of claim 33, wherein the salt is selected from the group consisting of (NH₄)₃PO₄, sodium formate, ammonium formate, K₂CO₃, KHCO₃, Na₂CO₃, NaHCO₃, MgSO₄, MgCO₃, CaCO₃, CsOH, Cs₂CO₃, Ba(OH)₂, and BaCO₃.

40. The method of claim 33, wherein the salt is selected from the group consisting of NH₄Cl, NH₄OH, tetramethyl ammonium chloride, tetrabutyl ammonium chloride, tetramethyl ammonium hydroxide, and tetrabutyl ammonium hydroxide.

41. The method of claim 24, wherein said surfactant dissolves in the aqueous solution at a concentration of 0.05%-10% (w/w).

42. The method of claim 41, wherein the surfactant is selected from the group consisting of anionic surfactant, nonionic surfactant, cationic surfactant, and amphoteric surfactant;
wherein the anionic surfactant is carboxylates, sulphonates, petroleum sulphonates, alkylbenzenesulphonates, naphthalenesulphonates, olefin sulphonates, alkyl sulphates, sulphates, sulphated natural oils, sulphated natural fats, sulphated esters, sulphated alkanolamides, sulphated alkylphenols, ethoxylated alkylphenols, or sodium N-lauroyl sarcosinate (NLS);
wherein the nonionic surfactant is ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, or polyoxyethylene fatty acid amides;
wherein the cationic surfactant is quaternary ammonium salts, amines with amide linkages, polyoxyethylene alkyl amines, polyoxyethylene alicyclic amines, n,n,n′,n′ tetrakis substituted ethylenediamines, or 2-alkyl 1-hydroxethyl 2-imidazolines;
wherein the amphoteric surfactant is n-coco 3-aminopropionic acid or sodium salt thereof, n-tallow 3-iminodipropionate or disodium salt thereof, n-carboxymethyl n dimethyl n-9 octadecenyl ammonium hydroxide, or n-cocoamidethyl n hydroxyethylglycine or sodium salt thereof.

43. The method of claim 41, wherein the surfactant is selected from the group consisting of Triton X-100, Triton X-114, Triton X-45, Tween 20, Igepal CA630, Brij 58, Brij O10, Brij L23, Pluronic L-61, Pluronic F-127, sodium dodecyl sulfate, sodium cholate, sodium deoxycholate, N-lauroyl sarcosine sodium salt, Hexadecyltrimethlammonium bromide, and span 80.

44. The method of claim 4, wherein the at least one chaotropic agent of the binding buffer comprises an anion selected from the group consisting of thiocyanate, isothiocyanate, perchlorate, acetate, trichloroacetate, trifluoroacetate, chloride, and iodide.

45. The method of claim 4, wherein the at least one chaotropic agent of the binding buffer is selected from the group consisting of guanidinium hydrochloride (GHCl), guanidinium thiocyanate, guanidinium isothiocyanate (GITC), sodium thiocyanate, sodium iodide, sodium perchlorate, sodium trichloroacetate, sodium trifluroacetate, lithium perchlorate, lithium acetate, magnesium chloride, phenol, 2-propanol, thiourea, and urea.

46. The method of claim 5, wherein the at least one chaotropic agent of the target binding buffer comprises an anion selected from the group consisting of thiocyanate, isothiocyanate, perchlorate, acetate, trichloroacetate, trifluoroacetate, chloride, and iodide.

47. The method of claim 5, wherein the at least one chaotropic agent of the target binding buffer is selected from the group consisting of guanidinium hydrochloride (GHCl), guanidinium thiocyanate, guanidinium isothiocyanate (GITC), sodium thiocyanate, sodium iodide, sodium perchlorate, sodium trichloroacetate, sodium trifluroacetate, lithium perchlorate, lithium acetate, magnesium chloride, phenol, 2-propanol, thiourea, and urea.

48. The method of claim 1, wherein the sample is blood, plasma, urine, saliva, stool, cerebrospinal fluid (CSF), lymph, serum, sputum, peritoneal fluid, sweat, tears, nasal swab, vaginal swab, endocervical swab, semen, or breast milk.

49. The method of claim 1, wherein the step (a) comprises the step of preparing a DNA library from the sample, resulting in the sample solution.

50. The method of claim 1, wherein the target nucleic acids are cell-free DNA and circulating tumor DNA, whereby the method increases a ratio of circulating tumor DNA:cell-free DNA, and/or variant allele frequency (VAF) in the sample for cancer diagnostic assay.

51. The method of claim 1, wherein the target nucleic acids are circulating fetal DNA, whereby the method enriches fetal fraction in the sample for non-invasive prenatal testing.

52. A kit for isolating target nucleic acids below a target size from a sample comprising nucleic acid components, comprising:
(a) at least one ATPS components selected from the group consisting of a polymer, salt, surfactant, and combinations thereof;
(b) a plurality of beads;
(c) a fractionation buffer comprising at least one chaotropic agent selected from the group consisting of thiocyanate, isothiocyanate, perchlorate, acetate, trichloroacetate, trifluoroacetate, chloride, and iodide; and
(d) a binding buffer comprises at least one chaotropic agent selected from the group consisting of thiocyanate, isothiocyanate, perchlorate, acetate, trichloroacetate, trifluoroacetate, chloride, and iodide.

53. The kit of claim 52, wherein the plurality of beads is magnetic beads, silica-based beads, carboxyl beads, hydroxyl beads, amine-coated beads, or any combination thereof.

54. The kit of claim 52, wherein the at least one chaotropic agent of the fractionation buffer is selected from the group consisting of guanidinium hydrochloride (GHCl), guanidinium thiocyanate, guanidinium isothiocyanate (GITC), sodium thiocyanate, sodium iodide, sodium perchlorate, sodium trichloroacetate, sodium trifluroacetate, lithium perchlorate, lithium acetate, magnesium chloride, phenol, 2-propanol, thiourea, and urea.

55. The kit of claim 52, wherein the at least one chaotropic agent has a concentration of around 1.5-8M in the fractionation buffer.

56. The kit of claim 52, wherein the fractionation buffer further comprises at least one polymer selected from the group consisting of polyvinyl alcohol, polyethylene glycol, polypropylene glycol, dextran, poly(ethylene glycol-ran-propylene glycol), pluronics, polyvinylpyrolidone, and polyacrylate.

57. The kit of claim 56, wherein the at least one polymer is present at a concentration of around 0.1-15% (w/w) in the fractionation buffer.

58. The kit of claim 52, wherein the fractionation buffer further comprises one or more of a pH buffer, metal chelator, or combination thereof.
